(19) United States
(12) Patent Application Publication
(10) Pub. No.: US 2025/0136709 A1
(43) Pub. Date: May 1, 2025

(54) ANTI-CD73 ANTIBODIES AND USE THEREOF

(71) Applicant: Development Center for Biotechnology, New Taipei City (TW)

(72) Inventors: Chun-Chung LEE, Taipei City (TW); Yu-Hsun LO, Taipei City (TW); Chu-Bin LIAO, Taipei City (TW); Chen-Jei HONG, Taipei City (TW); Sih-Yu CHEN, Taipei City (TW); Yen-Yu WU, Taipei City (TW); Szu-Liang LAI, Taipei City (TW); Chih-Yung HU, Taipei City (TW); Wen-Bin KE, Taipei City (TW); Ya-Ting JUAN, Taipei City (TW); Kao-Jean HUANG, Taipei City (TW)

(21) Appl. No.: 18/944,819

(22) Filed: Nov. 12, 2024

Related U.S. Application Data
(63) Continuation of application No. 17/686,666, filed on Mar. 4, 2022, now Pat. No. 12,187,806.

(51) Int. Cl. C07K 16/28; A61K 39/00; A61K 39/395; A61K 45/06; A61P 35/00

(52) U.S. Cl. CPC C07K 16/2896; A61K 39/39558; A61K 45/06; A61P 35/00; A61K 2039/505; C07K 2317/24; C07K 2317/34; C07K 2317/622; C07K 2317/75; C07K 2317/76; C07K 2317/92

(57) ABSTRACT

The present invention relates to a novel antibody, an antigen-binding fragment thereof and the uses of the antibody and fragment, wherein the antibody and the fragment comprise specific complementarity-determining regions (CDRs) and/or specifically bind to human CD73 at specific epitopes.

PRIORITY CLAIM

This application is a continuation of U.S. patent application Ser. No. 17/686,666, filed Mar. 4, 2022, the subject matter of which is incorporated herein in its entirety by reference.

### CROSS REFERENCE TO SEQUENCE LISTING

The present application incorporates by reference the Sequence Listing named “Sequence_Listing_XML”, created Jan. 7, 2025, with the size of 50,992 bytes.

### FIELD OF THE INVENTION

The present invention relates to novel antibodies, antigen-binding fragments of the antibodies and uses of the antibodies and fragments, wherein the antibodies and the fragments comprise specific complementarity-determining regions (CDRs) and/or specifically bind to human CD73 at specific epitopes.

### BACKGROUND OF THE INVENTION

The immunosuppressive effects in the tumor microenvironment are closely related to the progression of tumor growth. Tumor cells can change the microenvironment to escape immune surveillance. In recent research, several tumor-induced immune escape mechanisms have been found, wherein the co-inhibitory receptors (also called immune checkpoints, such as CTLA-4, PD-1) expressed on the T-cell surface have been taken as the targets of immunotherapy and successfully developed as immune checkpoint-inhibiting drugs.

During inflammation, adenosine triphosphate (ATP) is released from inflammatory cells, and the extracellular ATP attracts dendritic cells, macrophages or neutrophils via binding to the purinergic receptors (e.g., P2X receptor and P2Y receptor) expressed on the surface of these immune cells and causing immune responses. Moreover, extracellular ATP can also act as a source of immunosuppressant adenosine via CD39 and CD73 mediated hydrolysis. When inflammation is over, the membrane protein CD39 will convert ATP into adenosine diphosphate (ADP) or adenosine monophosphate (AMP) and the membrane protein CD73 will convert AMP into adenosine. Adenosine can inhibit the immune responses by binding to the receptors of immune cells (especially A2A and A2B receptors) and promoting the synthesis of cAMP that protect tissue from over inflammation. Beyond the role in regulation of immune response, the immunosuppressive effect of adenosine has also been recently reported as a critical factor that promote tumor growth. In the tumor microenvironment, the release of ATP and expression of CD39 and CD73 are increased due to hypoxia-induced tumor cells necrosis, and this causes an accumulation of adenosine. Furthermore, the expressions of A2A receptor and A2B receptor on immune cells are also increased due to the anoxic environment around the tumor. It has been proved that CD73 can promote the growth and angiogenesis of tumors, and the adenosine-induced immune-suppression, which are different from the other known mechanisms (e.g., suppressing immune response through immune-receptor tyrosine-based inhibition motif (ITIM)). The high expressions of CD73 and adenosine both are highly correlated with the progression and metastasis of tumors. Some inhibitors of adenosine signaling by targeting CD73 or adenosine A2A receptor, are expected to have a synergistic effect when used in combination with other immunotherapy drugs for cancer therapy. Therefore, for the purpose of cancer therapy, the drugs or methods targeting CD73 are under aggressive development for inhibiting the production of adenosine and/or the adenosine-induced immune-suppression.

Several kinds of anti-CD73 antibodies are undergoing phase 1 or phase 2 clinical trials, used alone or in combination with other drugs such as anti-PD-1, anti-PDL1, anti-CTLA4, EGFR inhibitor, and A2AR inhibitor (e.g., Ipilimumab and Nivolumab). For instance, Oleclumab (MEDI9447), a human anti-CD73 antibody, is currently being evaluated in a clinical trial. Nevertheless, there is still a necessity and urgency for developing novel anti-CD73 antibodies that can provide a better inhibitory effect on CD73.

### SUMMARY OF THE INVENTION

Inventors of the present invention discovered novel antibodies and the antigen-binding fragments thereof. The novel antibodies and antigen-binding fragments comprise specific complementarity-determining regions (CDRs) and can specifically bind to human CD73 at specific epitopes (e.g., the glutamic acid residue at position 296 and the arginine residue at position 297), and thus can inhibit CD73 activity and inhibit AMP consumption. Furthermore, compared to the positive control (i.e., Oleclumab), the antibodies and antigen-binding fragments of the present invention have a better and more long-term effective duration of inhibiting AMP consumption, and a better effect on activating immune cells and inhibiting tumor growth.

Therefore, an objective of the present invention is to provide an antibody or an antigen-binding fragment thereof, comprising: (i) a heavy chain variable domain comprising a HCDR1 region, a HCDR2 region, and a HCDR3 region, wherein the HCDR1 region comprises an amino acid sequence of SEQ ID NO: 4 with at most one variation, the HCDR2 region comprises an amino acid sequence of SEQ ID NO: 5 with at most one variation, and the HCDR3 region comprises an amino acid sequence of SEQ ID NO: 6 with at most one variation; and (ii) a light chain variable domain comprising a LCDR1 region, a LCDR2 region, and a LCDR3 region, wherein the LCDR1 region comprises an amino acid sequence of SEQ ID NO: 7 with at most one variation, the LCDR2 region comprises an amino acid sequence of SEQ ID NO: 8 with at most one variation, and the LCDR3 region comprises an amino acid sequence of SEQ ID NO: 9 with at most one variation.

Preferably, in the antibody or antigen-binding fragment thereof in accordance with the present invention as described above, the HCDR1 region comprises an amino acid sequence of SEQ ID NO: 4 with at most one variation, the HCDR2 region comprises an amino acid sequence of SEQ ID NO: 5 with at most one variation, and the HCDR3 region comprises an amino acid sequence of SEQ ID NO: 6 with at most one variation; and the LCDR1 region comprises an amino acid sequence of SEQ ID NO: 7, the LCDR2 region comprises an amino acid sequence of SEQ ID NO: 8, and the LCDR3 region comprises an amino acid sequence of SEQ ID NO: 9 with at most one variation.

In certain embodiments of the antibody or antigen-binding fragment thereof in accordance with the present invention as described above, the HCDR1 region comprises an amino acid sequence of SEQ ID NO: 4 with variation at position 8; the HCDR2 region comprises an amino acid sequence of SEQ ID NO: 5 with variation at position 1; the HCDR3 region comprises an amino acid sequence of SEQ ID NO: 6 with variation at position 1; the LCDR1 region comprises an amino acid sequence of SEQ ID NO: 7, the LCDR2 region comprises an amino acid sequence of SEQ ID NO: 8; and the LCDR3 region comprises an amino acid sequence of SEQ ID NO: 9 with variation at position 8. Preferably, the HCDR1 region comprises an amino acid sequence of SEQ ID NO: 4, wherein the tryptophan residue at position 8 is substituted by threonine; the HCDR2 region comprises an amino acid sequence of SEQ ID NO: 5, wherein the glutamine residue at position 1 is substituted by serine, arginine, threonine or histidine, the HCDR3 region comprises an amino acid sequence of SEQ ID NO: 6, wherein the phenylalanine residue at position 1 is substituted by leucine, tyrosine or isoleucine; and the LCDR3 region comprises an amino acid sequence of SEQ ID NO: 9, wherein the leucine residue at position 8 is substituted by methionine, glycine, histidine, arginine, glutamine or isoleucine.

In certain embodiments of the antibody or antigen-binding fragment thereof in accordance with the present invention as described above, the HCDR1 region comprises an amino acid sequence selected from the group consisting of SEQ ID NO: 4 and SEQ ID NO: 15; the HCDR2 region comprises an amino acid sequence selected from the group consisting of SEQ ID NO: 5, SEQ ID NO: 16 to SEQ ID NO: 19; and the HCDR3 region comprises an amino acid sequence selected from the group consisting of SEQ ID NO:6 (FAD); SEQ ID NO: 20 (LAD); SEQ ID NO: 21 (YAD) and SEQ ID NO: 22 (IAD). In certain embodiments of the antibody or antigen-binding fragment thereof in accordance with the present invention as described above, the LCDR1 region comprises an amino acid sequence of SEQ ID NO: 7; the LCDR2 region comprises an amino acid sequence of SEQ ID NO: 8; and the LCDR3 region comprises an amino acid sequence selected from the group consisting of SEQ ID NO: 9, SEQ ID NO: 23 to SEQ ID NO: 28.

In certain embodiments of the antibody or antigen-binding fragment thereof in accordance with the present invention as described above, the heavy chain variable domain comprises an amino acid sequence selected from the group consisting of: (i) an amino acid sequence having at least 95% identity to SEQ ID NO: 2 (i.e., 10H5 chimeric Heavy chain), (ii) an amino acid sequence having at least 95% identity to SEQ ID NO: 10 (i.e., 10H5 humanized Heavy chain HuB9), and (iii) an amino acid sequence having at least 95% identity to SEQ ID NO: 11 (i.e., 10H5 humanized Heavy chain HuB10). More preferably, the heavy chain variable domain comprises an amino acid sequence selected from the group consisting of SEQ ID NO: 2, SEQ ID NO: 10, SEQ ID NO: 11 and SEQ ID NO: 29 to SEQ ID NO: 38.

In certain embodiments of the antibody or antigen-binding fragment thereof in accordance with the present invention as described above, the light chain variable domain comprises an amino acid sequence selected from the group consisting of: (i) an amino acid sequence having at least 95% identity to SEQ ID NO: 3 (i.e., 10H5 chimeric Light chain), (ii) an amino acid sequence having at least 95% identity to SEQ ID NO: 12 (i.e., 10H5 humanized Light chain HdB6), and (iii) an amino acid sequence having at least 95% identity to SEQ ID NO: 13 (i.e., 10H5 humanized Light chain HdB7). More preferably, the light chain variable domain comprises an amino acid sequence selected from the group consisting of SEQ ID NO: 3, SEQ ID NO: 12, SEQ ID NO: 13 and SEQ ID NO: 39 to SEQ ID NO: 44.

In certain embodiments of the present invention, the antibody or antigen-binding fragment thereof as described above further comprises one or more of (1) a linker peptide between the heavy chain variable domain and the light chain variable domain, (2) a heavy chain constant region, (3) a light chain constant region, and (4) an Fc region.

Preferably, the antibody or antigen-binding fragment thereof in accordance with the present invention as described above is a single chain antibody fragment, a bispecific antibody, a single-domain antibody, a nanobody, a chimeric antibody, or a partially or fully humanized antibody. More preferably, the antibody or antigen-binding fragment thereof further links with a drug conjugate to form an antibody-drug conjugate (ADC), or further links with a second antibody or a second antigen binding fragment to form a bispecific antibody.

In various embodiments of the present invention, the heavy chain constant region or fragment thereof is an IgG constant region, including for example an IgG1 constant region, an IgG2 constant region, an IgG3 constant region or an IgG4 constant region. In one embodiment, the antibody or antigen-binding fragment thereof further comprises a fragment derived from IgG4.

In various embodiments of any aspect delineated herein, the IgG constant region has one or more amino acid substitutions relative to a wild-type IgG constant region where the modified IgG has an increased half-life compared to the half-life of an IgG having the wild-type IgG constant region.

In various embodiments of any aspect delineated herein, the IgG constant region has one or more amino acid substitutions of amino acid residues at positions 251-257, 285-290, 308-314, 385-389, and 428-436, where the numbering is according to the EU index as set forth in Kabat. In various embodiments of any aspect delineated herein, the antigen-binding fragment is Fv, Fab, F(ab′)2, Fab′, dsFv, scFv, or sc(Fv)2.

In certain embodiments of the present invention, the antibody or antigen-binding fragment thereof as described above further comprises a fragment derived from IgG, IgM, IgA, IgE or IgD.

In certain embodiments of the present invention, the antibody or antigen-binding fragment thereof as described above specifically binds to CD73. Preferably, the antibody or antigen-binding fragment thereof specifically binds to CD73 comprising an amino acid sequence of SEQ ID NO: 14 (i.e., human CD73 269-304). More preferably, the antibody or antigen-binding fragment thereof specifically binds to CD73 comprising an amino acid sequence of SEQ ID NO: 1 (i.e., human CD73).

In certain embodiments of the present invention, the antibody or antigen-binding fragment thereof as described above binds to the CD73 on at least one of the glutamic acid residue at position 296 and the arginine residue at position 297. Preferably, the antibody or antigen-binding fragment thereof binds to the CD73 on both the glutamic acid residue at position 296 and the arginine residue at position 297.

Another objective of the present invention is to provide an antibody or an antigen-binding fragment thereof, wherein the antibody or antigen-binding fragment thereof specifically binds to human CD73 on at least one of the glutamic acid residue at position 296 and the arginine residue at position 297. Preferably, the antibody or an antigen-binding fragment thereof binds to human CD73 on both the glutamic acid residue at position 296 and the arginine residue at position 297.

Preferably, in the antibody or an antigen-binding fragment thereof in accordance with the present invention that specifically binds to human CD73 on at least one of the glutamic acid residue at position 296 and the arginine residue at position 297, the CD73 comprises an amino acid sequence of SEQ ID NO: 14 (i.e., human CD73 269-304). More preferably, the CD73 comprises an amino acid sequence of SEQ ID NO: 1 (i.e., human CD73).

Preferably, the antibody or antigen-binding fragment thereof in accordance with the present invention that specifically binds to human CD73 on at least one of the glutamic acid residue at position 296 and the arginine residue at position 297 is a single chain antibody fragment, a bispecific antibody, a single-domain antibody, a nanobody, a chimeric antibody, or a partially or fully humanized antibody. More preferably, the antibody or antigen-binding fragment thereof that specifically binds to human CD73 on at least one of the glutamic acid residue at position 296 and the arginine residue at position 297 further links with a drug conjugate to form an antibody-drug conjugate (ADC), or further links with a second antibody or a second antigen binding fragment to form a bispecific antibody.

In certain embodiments of the antibody or an antigen-binding fragment thereof in accordance with the present invention that specifically binds to human CD73 on at least one of the glutamic acid residue at position 296 and the arginine residue at position 297, the antibody or antigen-binding fragment thereof further comprises a fragment derived from IgG1, IgG2, IgG3 or IgG4. In one embodiment of the antibody or an antigen-binding fragment thereof that specifically binds to human CD73 on at least one of the glutamic acid residue at position 296 and the arginine residue at position 297, the antibody or antigen-binding fragment thereof further comprises a fragment derived from IgG4.

Still another objective of the present invention is to provide a nucleic acid molecule encoding the antibody or antigen-binding fragment thereof as described above.

Yet another objective of the present invention is to provide a vector comprising the nucleic acid molecule as described above.

Yet another objective of the present invention is to provide a recombinant host cell comprising a nucleic acid molecule and/or a vector as described above.

Yet another objective of the present invention is to provide a pharmaceutical composition comprising (i) an antibody or antigen-binding fragment thereof as described above, a nucleic acid molecule encoding the aforesaid antibody or antigen-binding fragment thereof, a vector comprising the aforesaid nucleic acid molecule, a recombinant host cell comprising the aforesaid nucleic acid molecule, or a recombinant host cell comprising the aforesaid vector, and (ii) a pharmaceutically acceptable carrier. In certain embodiments of the present invention, the pharmaceutical composition is used for inhibiting CD73. In certain embodiments of the present invention, the pharmaceutical composition further comprises one or more other immunotherapy agents.

Yet another objective of the present invention is to provide a pharmaceutical composition for use in inhibiting CD73 in a subject in need thereof, wherein the pharmaceutical composition comprises (i) an antibody or antigen-binding fragment thereof as described above, a nucleic acid molecule encoding the aforesaid antibody or antigen-binding fragment thereof, a vector comprising the aforesaid nucleic acid molecule, a recombinant host cell comprising the aforesaid nucleic acid molecule, or a recombinant host cell comprising the aforesaid vector, and (ii) a pharmaceutically acceptable carrier. In certain embodiments of the pharmaceutical composition for use in inhibiting CD73 of the present invention, the pharmaceutical composition is administered in combination with one or more other immunotherapy agents.

Preferably, the other immunotherapy agent used in the present invention is PD-1 antagonist, PD-L1 antagonist or CTLA-4 antagonist.

Preferably, the pharmaceutical composition in accordance with the present invention is used for activating T-cells, activating B-cells, activating NK cells, and/or inhibiting cancer cells.

Preferably, the pharmaceutical composition in accordance with the present invention is used for treating, ameliorating and/or preventing cancer. More preferably, the cancer is selected from the group consisting of breast cancer, gastric cancer, colorectal cancer, gallbladder cancer, prostate cancer, ovarian carcinoma, chronic or acute lymphocytic leukemia, bladder cancer, brain tumor, kidney carcinoma, head and neck squamous cell carcinoma, glioblastoma, esophageal cancer, pancreatic cancer, renal carcinoma, oral cancer, lung cancer, colon adenocarcinoma, melanoma and lymphoma.

Yet another objective of the present invention is to provide a method of inhibiting CD73, comprising administering a subject in need thereof an effective amount of a pharmaceutical composition as described above. Preferably, the method in accordance with the present invention further comprises administering one or more other immunotherapy agents to the subject in need thereof. More preferably, the other immunotherapy agent is PD-1 antagonist, PD-L1 antagonist or CTLA-4 antagonist.

Preferably, the method in accordance with the present invention is for activating T-cells, activating B-cells, activating NK cells and/or inhibiting cancer cells.

Preferably, the method in accordance with the present invention is for treating, ameliorating and/or preventing cancer. More preferably, the cancer is selected from the group consisting of breast cancer, gastric cancer, colorectal cancer, gallbladder cancer, prostate cancer, ovarian carcinoma, chronic or acute lymphocytic leukemia, bladder cancer, brain tumor, kidney carcinoma, head and neck squamous cell carcinoma, glioblastoma, esophageal cancer, pancreatic cancer, renal carcinoma, oral cancer, lung cancer, colon adenocarcinoma, melanoma, and lymphoma.

Yet another objective of the present invention is to provide use of the antibody or antigen-binding fragment thereof as described above in the manufacture of a medicament, wherein the medicament is for inhibiting CD73. Preferably, the medicament is used in combination with one or more other immunotherapy agents. More preferably, the other immunotherapy agent is PD-1 antagonist, PD-L1 antagonist, or CTLA-4 antagonist.

Preferably, the medicament is used for activating T-cells, activating B-cells, activating NK cells and/or inhibiting cancer cells.

Preferably, the medicament is used for treating, ameliorating, and/or preventing cancer. More preferably, the cancer is selected from the group consisting of breast cancer, gastric cancer, colorectal cancer, gallbladder cancer, prostate cancer, ovarian carcinoma, chronic or acute lymphocytic leukemia, bladder cancer, brain tumor, kidney carcinoma, head and neck squamous cell carcinoma, glioblastoma, esophageal cancer, pancreatic cancer, renal carcinoma, oral cancer, lung cancer, colon adenocarcinoma, melanoma, and lymphoma.

Yet another objective of the present invention is to provide use of the antibody or antigen-binding fragment thereof as described above for inhibiting CD73. Preferably, the antibody or antigen-binding fragment thereof is used in combination with one or more other immunotherapy agents. More preferably, the other immunotherapy agent is PD-1 antagonist, PD-L1 antagonist, or CTLA-4 antagonist.

Preferably, the antibody or antigen-binding fragment thereof is used for activating T-cells, activating B-cells, activating NK cells and/or inhibiting cancer cells.

Preferably, the antibody or antigen-binding fragment thereof is used for treating, ameliorating and/or preventing cancer. More preferably, the cancer is selected from the group consisting of breast cancer, gastric cancer, colorectal cancer, gallbladder cancer, prostate cancer, ovarian carcinoma, chronic or acute lymphocytic leukemia, bladder cancer, brain tumor, kidney carcinoma, head and neck squamous cell carcinoma, glioblastoma, esophageal cancer, pancreatic cancer, renal carcinoma, oral cancer, lung cancer, colon adenocarcinoma, melanoma and lymphoma.

### BRIEF DESCRIPTION OF THE DRAWINGS

The patent application contains at least one drawing executed in color. Copies of this patent with color drawing(s) will be provided by the Patent and Trademark Office upon request and payment of the necessary fee.

FIG. 1A indicates that the antibody of present invention, 10H5, has a superior effect on inhibiting CD73 activity than reference antibody, Oleclumab, in MDA-MB-231 triple negative breast cancer (TNBC) cells. FIG. 1B indicates that the antibody 10H5 also has a superior effect on inhibiting CD73 activity than Oleclumab in Calu-1, a non-small cell lung cancer (NSCLC) cell line.

FIGS. 2A and 2B indicate that the antibody of present invention, 10H5 and its humanized derivatives, HuB9/HdB7, HuB10/HdB6 and HuB10/HdB7, have a superior effect on inhibiting CD73 activity than Oleclumab in different cancer cell types (FIG. 2A: MDA-MB-231 cells; FIG. 2B: Calu-1 cells).

FIGS. 3A to 3D show the Biacore SPR results of the binding affinity of the antibodies of the present invention, 10H5 (FIG. 3A) and its humanized derivatives, HuB9/HdB7 (FIG. 3B), HuB10/HdB6 (FIG. 3C) and HuB10/HdB7 (FIG. 3D), to human CD73 antigen.

FIGS. 4A and 4B indicate the antibodies of the present invention, 10H5 and its humanized derivatives, HuB9/HdB7, HuB10/HdB6 and HuB10/HdB7, have a more long-lasting inhibitory activity against CD73 compared to Oleclumab (FIG. 4A: MDA-MB-231 cells; FIG. 4B: Calu-1 cells).

FIG. 5 indicates the antibodies of the present invention, 10H5 and its humanized derivatives, HuB9/HdB7, HuB10/HdB6 and HuB10/HdB7, have a more long-lasting inhibitory activity against CD73 compared to Oleclumab by measuring the actual AMP concentration.

FIG. 6 indicates that the antibodies of the present invention, 10H5 and its humanized derivatives, HuB9/HdB7, HuB10/HdB6 and HuB10/HdB7, are more effective than Oleclumab in reversing the T cell proliferation attenuated by exposure of AMP.

FIG. 7 indicates that the antibodies of the present invention, 10H5 and its humanized derivatives, HuB9/HdB7, HuB10/HdB6 and HuB10/HdB7, can inhibit MDA-MB-231 tumor growth in mouse xenograft model.

FIG. 8 shows the CD73 activity in tumor tissue was detected after the 24-hour treatment of the antibody of the present invention (e.g., 10H5), which was detected by in situ CD73 activity assay. The deep brown color indicates the presence of active CD73, whereas the light brown color indicates that CD73 enzymatic activity was inhibited by the increase dose of 10H5.

FIG. 9 shows the design of domain swapping of mouse CD73 protein for identifying the epitopes of the antibodies of the present invention.

FIG. 10 indicates that 10H5 has species specificity and binds to the human CD73 on the amino acids at positions 269-304, which is different from the binding sites of Oleclumab (i.e., K206 and N211).

FIG. 11 shows the amino acid residue at positions that are different between mouse CD73 and human CD73, and can be mutated for mapping the binding positions of the antibodies of the present invention.

FIG. 12 indicates that the antibodies of the present invention (e.g., 10H5) bind to the CD73 depending on the glutamic acid residue at position 296 and the arginine residue at position 297.

FIG. 13 shows the ELISA results, representing the binding affinity of the antibodies of the present invention to mouse CD73-D296E-K297R, to confirm that the antibodies of the present invention (e.g., 10H5) bind to the CD73 depending on the glutamic acid residue at position 296 and the arginine residue at position 297.

FIG. 14A shows a 3D structure model, simulating the interactions between human CD73 and 10H5 scFv antibody. FIG. 14B shows that 10H5 scFv antibody binds to the human CD73 on glutamic acid residue at position 296 and the arginine residue at position 297. FIG. 14C shows that there are hydrogen bonds, hydrophobic effect, and ion-ion interaction between the six amino acids (W33 of CDR-H1, N35 of CDR-H1, Q50 of CDR-H2, F95 of CDR-H3, Y91 of CDR-L3 and L96 of CDR-L3) of 10H5 scFv antibody and human CD73 (on glutamic acid residue at position 296 and the arginine residue at position 297).

FIG. 15 shows that 4 positions: W33 of CDR-H1, N35 of CDR-H1, F95 of CDR-H3, and Y91 of CDR-L3 of 10H5 antibody were respectively mutated to alanine for mapping the binding positions of the antibodies of the present invention, wherein the results show that 10H5 could not bind to the mouse CD73 (left figure), but could bind to the mouse CD73 containing hF1 (human CD73 residues 269-304) (right figure). However, all of the four mutated antibodies could not bind to mouse CD73 hF1 (right figure). These results confirm that the 3D structure model can simulate the interactions between CD73 and 10H5 antibody.

FIG. 16 shows the mutation position in the CDR sequences and the binding affinity (K_{D}) to the mouse CD73 D296E-K297R of the 16 antibodies, which were selected from the phage display library constructed according to 3D modeling simulation.

FIGS. 17A and 17B indicate that all the 16 antibodies selected from the phage display library and were constructed into hIgG4 type, as well as the 10H5 antibody and its humanized derivatives (i.e., HuB10/HdB6), can inhibit the CD73 activity of MDA-MB-231 cells.

### DESCRIPTION OF THE PREFERRED EMBODIMENTS

The following paragraphs will describe some of the embodiments of the present invention in detail. However, without departing from the spirit of the present invention, the present invention may be embodied in various embodiments and should not be limited to the embodiments described in the specification or defined in the appended claims.

Unless otherwise indicated herein, the expression “a,” “an,” “the,” or the like recited in the specification of the present invention (especially in the claims) are intended to include both the singular and plural forms; the term “subject” recited herein refers to human or non-human mammalian (e.g., dog, cat).

The term “CD73” recited herein refers to primates CD73, such as human CD73. Human CD73 comprises an amino acid sequence of SEQ ID NO: 1, and the amino acid sequence of human CD73 at positions 269-304 is SEQ ID NO: 14.

It has been known that CD73 closely relates to the progression and prognosis of many types of cancers. The relationships between CD73 and cancers such as breast cancer (e.g., triple-negative breast cancer and invasive lobular breast cancer), gastric cancer, colorectal cancer, gallbladder cancer, prostate cancer, ovarian carcinoma, chronic or acute lymphocytic leukemia, bladder cancer, brain tumor, kidney carcinoma, head and neck squamous cell carcinoma, glioblastoma, esophageal cancer, pancreatic cancer, renal carcinoma, oral cancer (e.g., oral cavity squamous cell carcinoma), lung cancer (e.g., non-small cell lung cancer, lung large cell cancer, and lung adenocarcinoma), colon adenocarcinoma, melanoma, and lymphoma can be seen in the publications listed below:

| Cancer | Publication |
|---|---|
| Breast cancer | Loi S, Pommey S, Haibe-Kains B, et al. CD73 promotes anthracycline |
| | resistance and poor prognosis in triple negative breast cancer. Proc Nat |
| | Acad Sci. 2013; 110(27): 11091-11096. |
| | Tao Jiang et al. Comprehensive evaluation of NT5E/CD73 expression |
| | and its prognostic significance in distinct types of cancers. BMC Cancer. |
| | (2018) 18: 267. |
| Gastric cancer | Lu -X-X, Chen Y-T, Feng B, et al. Expression and clinical significance |
| | of CD73 and hypoxia-inducible factor-1α in gastric carcinoma. World J |
| | Gastroenterol. 2013; 19(12): 1912. |
| | Tao Jiang et al. Comprehensive evaluation of NT5E/CD73 expression |
| | and its prognostic significance in distinct types of cancers. BMC Cancer. |
| | (2018) 18: 267. |
| Colorectal cancer | Wu XR, He XS, Chen YF, et al. High expression of CD73 as a poor |
| | prognostic biomarker in human colorectal cancer. J Surg Oncol. |
| | 2012; 106(2): 130-137. |
| | Liu N, Fang XD, Vadis Q. CD73 as a novel prognostic biomarker for |
| | human colorectal cancer. J Surg Oncol. 2012; 106 (7): 918-919. |
| Gallbladder | Xiong L, Wen Y, Miao X, et al. NT5E and FcGBP as key regulators of |
| cancer patients | TGF-1-induced epithelial-mesenchymal transition (EMT) are associated |
| | with tumor progression and survival of patients with gallbladder cancer. |
| | Cell Tissue Res. 2014; 355(2): 365-374. |
| Prostate cancer | Yang Q, Du J, Zu L. Overexpression of CD73 in prostate cancer is |
| | associated with lymph node metastasis. Pathol Oncol Res. 2013 |
| | Oct; 19(4): 811-814. PubMed PMID: 23653114; eng. |
| | Tao Jiang et al. Comprehensive evaluation of NT5E/CD73 expression |
| | and its prognostic significance in distinct types of cancers. BMC Cancer. |
| | (2018) 18: 267. |
| Ovarian | Turcotte M, Spring K, Pommey S et al. CD73 is associated with poor |
| carcinoma | prognosis in high-grade 784 serous ovarian cancer. Cancer Res. |
| | doi: 10.1158/0008-5472. CAN-14-3569 (2015). |
| | Tao Jiang et al. Comprehensive evaluation of NT5E/CD73 expression |
| | and its prognostic significance in distinct types of cancers. BMC Cancer |
| | (2018) 18: 267. |
| Chronic | Serra S, Horenstein AL, Vaisitti T, et al. CD73-generated extracellular |
| lymphocytic | adenosine in chronic lymphocytic leukemia creates local conditions |
| leukemia | counteracting drug-induced cell death. Blood. 2011; 118 (23): 6141- |
| | 6152. |
| Acute | Coustan-Smith E, Song G, Clark C et al. New markers for minimal |
| lymphocytic | residual disease detection in 866 acute lymphoblastic leukemia. Blood. |
| leukemia | 117(23), 6267-6276 (2011). |
| Bladder cancer | Wettstein MS, Buser L, Hermanns T et al. CD73 predicts favorable |
| | prognosis in patients with 1080 nonmuscle-invasive urothelial bladder |
| | cancer. Disease Marker. 2015(1), 1-8 (2015) |
| Brain tumor | Xu S, Shao QQ, Sun JT et al. Synergy between the ectoenzymes CD39 |
| | and CD73 contributes to 882 adenosinergic immunosuppression in |
| | human malignant gliomas. Neuro Oncol. 15(9), 1160-1172 883 (2013). |
| Kidney carcinoma | Wettstein MS, Buser L, Hermanns T et al. CD73 predicts favorable |
| | prognosis in patients with 1080 nonmuscle-invasive urothelial bladder |
| | cancer. Disease Marker. 2015(1), 1-8 (2015) |
| Head and neck | CD73 is associated with poor prognosis in HNSCC Oncotarget. 2016 |
| squamous cell | Sep 20; 7(38): 61690-61702. |
| carcinoma | |
| Glioblastoma | Tao Jiang et al. Comprehensive evaluation of NT5E/CD73 expression |
| | and its prognostic significance in distinct types of cancers. BMC Cancer. |
| | (2018) 18: 267. |
| Esophageal | Tao Jiang et al. Comprehensive evaluation of NT5E/CD73 expression |
| cancer | and its prognostic significance in distinct types of cancers. BMC Cancer. |
| | (2018) 18: 267. |
| Pancreatic cancer | Tao Jiang et al. Comprehensive evaluation of NT5E/CD73 expression |
| | and its prognostic significance in distinct types of cancers. BMC Cancer. |
| | (2018) 18: 267. |
| Renal carcinoma | Tao Jiang et al. Comprehensive evaluation of NT5E/CD73 expression |
| | and its prognostic significance in distinct types of cancers. BMC Cancer. |
| | (2018) 18: 267. |
| Oral cancer | Tao Jiang et al. Comprehensive evaluation of NT5E/CD73 expression |
| | and its prognostic significance in distinct types of cancers. BMC Cancer. |
| | (2018) 18: 267. |
| Lung cancer | Tao Jiang et al. Comprehensive evaluation of NT5E/CD73 expression |
| | and its prognostic significance in distinct types of cancers. BMC Cancer. |
| | (2018) 18: 267. |
| Colon | Zhang B, et al. The expression and clinical significance of CD73 |
| adenocarcinoma | molecule in human rectal adenocarcinoma. Tumour Biol. |
| | 2015; 36(7): 5459-5466. |
| Melanoma | Wang H, Lee S, Nigro CL et al. NT5E (CD73) is epigenetically |
| | regulated in malignant melanoma 954 and associated with metastatic |
| | site specificity. Br J Cancer 106(8), 1446-1452 (2012). |
| | Tao Jiang et al. Comprehensive evaluation of NT5E/CD73 expression |
| | and its prognostic significance in distinct types of cancers. BMC Cancer. |
| | (2018) 18: 267. |
| Lymphoma | Wang X et al. Tumor CD73/A2aR adenosine immunosuppressive axis |
| | and tumor-infiltrating lymphocytes in diffuse large B-cell lymphoma: |
| | correlations with clinicopathological characteristics and clinical |
| | outcome Int J Cancer 2019 Sep 1; 145(5): 1414-1422. |

The term “antibody” recited herein includes a polyclonal antibody, a monoclonal antibody, a single chain antibody fragment, a bispecific antibody, a single-domain antibody, a nanobody, a chimeric antibody, or a partially or fully humanized antibody. The methods for generating an antibody reactive with a specific antigen are known in the art. For example, the antibody can be generated by recombinant methods, or by immunizing an animal with the antigen or an antigen-encoding nucleic acid.

A typical IgG antibody comprises two heavy chains and two light chains inter-connected by disulfide bonds. Each heavy chain has a constant domain (i.e., “C_{H}” or “CH”) and a variable domain (i.e., “V_{H}” or “VH”). Each light chain has a constant domain (i.e., “C_{L}” or “CL”) and a variable domain (i.e., “V_{L}” or “VL”). Each V_{H} and V_{L} is composed of three complementarity-determining regions (CDRs) and four framework regions (FRs), wherein the CDRs are hypervariable and the FRs are more conserved, arranged from amino-terminus to carboxy-terminus in the following order: FR1, CDR1, FR2, CDR2, FR3, CDR3, and FR4. The CDRs are primarily responsible for binding an epitope of an antigen. In certain embodiments of the present invention, the antibody comprises a fragment derived from IgG1, IgG2, IgG3, or IgG4. For example, in certain embodiments of the present invention, the antibodies comprise a fragment of the variable domain of IgG1, IgG2, IgG3, or IgG4.

The term “epitope” is the site on the antigen to which an antibody binds. US9938356B2 mentions an antibody that is capable of binding CD73 on epitopes such as positions 206 and 211 (The positions of 206 and 211 are the same positions of 180 and 185 described in US9938356B2 since the signal peptide sequence comprising 26 amino acids in the CD73 sequence was removed). Different from US9938356B2, the antibodies of the present invention bind to the CD73 on at least one of the glutamic acid residue at position 296 and the arginine residue at position 297.

The positive control (i.e., Oleclumab) used in the embodiments described in this specification was synthesized by the inventors and has an amino acid sequence the same as that of MEDI9447 disclosed by US9938356B2.

The term “antigen-binding fragment” of an antibody, and the like, as used herein, includes any naturally occurring, enzymatically obtainable, synthetic, or genetically engineered polypeptide or glycoprotein that specifically binds an antigen to form a complex. An antigen-binding fragment of an antibody may be derived, e.g., from a full antibody molecule using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

An antigen-binding fragment of an antibody typically comprises at least one variable domain. The variable domain may be of any size and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL, or VL-VL dimers. A linker may consist of at least 2 (e.g., 5, 10, 15, 20, 40, 60, or more) amino acids, and thus result in a flexible or semi-flexible linkage between adjacent VH and/or VL domain in a single polypeptide molecule. Alternatively, the antigen-binding fragment of an antibody may contain a monomeric VH or VL domain.

In certain embodiments, an antigen-binding fragment of an antibody may contain at least one variable domain covalently linked to at least one constant domain, and the variable and constant domains may be either directly linked to one another or can be linked by a linker. A linker may consist of at least 2 (e.g., 5, 10, 15, 20, 40, 60, or more) amino acids, and thus result in a flexible or semi-flexible linkage between adjacent variable and/or constant domains in a single polypeptide molecule. Moreover, the antigen-binding fragment of an antibody may comprise a homo-dimer or hetero-dimer (or other multimers) of any of the variable and constant domain configurations in non-covalent association with one another and/or with one or more monomeric VH or VL domain (e.g., by a disulfide bond(s)).

In certain embodiments, the antibody or antigen-binding fragment thereof in accordance with the present invention may also link with a drug conjugate to form an antibody-drug conjugate (ADC), or further links with a second antibody or a second antigen binding fragment to form a bispecific antibody.

In other embodiments, the antibody in accordance with the invention is a humanized antibody. A “humanized antibody” refers to a recombinant protein in which the CDRs from an antibody from one species (e.g., a rodent antibody) are transferred from the heavy and light chains of the species (rodent antibody) into human heavy and light domains, including human framework region (FR) sequences. The constant domains of the antibody molecule are derived from those of a human antibody.

The term “% identity” as used herein is defined as the percentage of amino acid residues or nucleotides in a candidate sequence that is identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity.

As mentioned above, inventors of the present invention found novel anti-CD73 antibodies that comprise specific complementarity-determining regions (CDRs) and are effective in inhibiting CD73. As compared to the positive control (i.e., Oleclumab), the antibodies and antigen-binding fragments in accordance with the present invention have a better CD73-binding activity, a longer effective duration of inhibiting AMP consumption, and a better effect on activating immune cells and inhibiting tumor growth.

Therefore, the present invention relates to a novel antibody or an antigen-binding fragment thereof, comprising: (i) a heavy chain variable domain comprising a HCDR1 region, a HCDR2 region, and a HCDR3 region, wherein the HCDR1 region comprises an amino acid sequence of SEQ ID NO: 4 with at most one variation, the HCDR2 region comprises an amino acid sequence of SEQ ID NO: 5 with at most one variation, and the HCDR3 region comprises an amino acid sequence of SEQ ID NO: 6 with at most one variation; and; and (ii) a light chain variable domain comprising a LCDR1 region, a LCDR2 region, and a LCDR3 region, wherein the LCDR1 region comprises an amino acid sequence of SEQ ID NO: 7 with at most one variation, the LCDR2 region comprises an amino acid sequence of SEQ ID NO: 8 with at most one variation, and the LCDR3 region comprises an amino acid sequence of SEQ ID NO: 9 with at most one variation.

Inventors of the present invention also found that the antibodies and antigen-binding fragments in accordance with the present invention can specifically bind to human CD73 on at least one of the glutamic acid residue at position 296 and the arginine residue at position 297. Accordingly, the present invention also relates to a novel antibody or an antigen-binding fragment thereof that specifically binds to human CD73 on at least one of the glutamic acid residue at position 296 and the arginine residue at position 297.

The present invention also relates to a nucleic acid molecule encoding the antibody or antigen-binding fragment thereof as described above; a vector comprising the nucleic acid molecule as described above; a recombinant host cell comprising a nucleic acid molecule and/or a vector as described above; a pharmaceutical composition comprising (i) an antibody or antigen-binding fragment thereof as described above, a nucleic acid molecule encoding the aforesaid antibody or antigen-binding fragment thereof, a vector comprising the aforesaid nucleic acid molecule, a recombinant host cell comprising the aforesaid nucleic acid molecule, or a recombinant host cell comprising the aforesaid vector, and (ii) a pharmaceutically acceptable carrier; a method of inhibiting CD73, comprising administering a subject in need thereof an effective amount of the pharmaceutical composition as described above; use of the antibody or antigen-binding fragment thereof as described above in the manufacture of a medicament for inhibiting CD73; and use of the antibody or antigen-binding fragment thereof as described above for inhibiting CD73.

The pharmaceutical composition or medicament in accordance with the present invention can be used for systemic or topical administration and can be delivered by various drug delivery systems (DDSs), such as an oral drug delivery system, a transdermal drug delivery system, a transmucosal drug delivery system, or an injectable drug delivery system. For example, to enhance bioavailability, control drug release speed, target the lesion precisely, and/or reduce side effects, the pharmaceutical composition or medicament can be delivered by a liposome, a microcapsule, nanoparticles, or microneedles, but is not limited thereby.

Depending on the desired purpose(s), the pharmaceutical composition or medicament in accordance with the present invention can be provided in any suitable form without particular limitations. For example, the pharmaceutical composition or medicament can be provided in a form for oral administration, intravenous injection (including drip infusion and bolus injection), intramuscular injection, subcutaneous injection, intraarterial injection, intraperitoneal injection, transdermal administration (such as a patch), or transmucosal administration (such as nasal spray, nasal drops, and suppository), but is not limited thereby. Depending on the form and purpose(s), a suitable carrier can be chosen and used to provide the pharmaceutical composition or medicament, wherein the carriers are known in the art of pharmaceutical engineering. Examples of the carrier include, but are not limited to, excipients, diluents, fillers, buffers, auxiliaries, stabilizers, absorption enhancers, disintegrating agents, hydrotropic agents, antioxidants, adhesives, binders, tackifiers, dispersants, suspending agents, lubricants, and hygroscopic agents.

As a form for oral administration, the pharmaceutical composition or medicament in accordance with the present invention can be provided by any suitable methods in any suitable form for oral administration, wherein the liquid form suitable for oral administration includes syrups, an oral solution, a suspension, and an elixir, and the solid form suitable for oral administration includes a powder, a granule, a troche, a dragee, an enteric-coated tablet, a chewable tablet, an effervescent tablet, a film-coated tablet, a capsule, and a long-acting slow-release tablet. The pharmaceutical composition or medicament provided in accordance with the present invention can comprise any pharmaceutically acceptable carrier that will not adversely affect the desired effects of the active ingredient (i.e., antibodies of the present invention or antigen-binding fragment thereof). For example, the pharmaceutically acceptable carriers for the aforesaid liquid form include but are not limited to, water, saline, dextrose, glycerol, ethanol or its analogs, oil (e.g., olive oil, castor oil, cottonseed oil, peanut oil, corn oil, and germ oil), glycerol, polyethylene glycol, and combinations thereof; and the pharmaceutically acceptable carriers for the aforesaid solid form include, but are not limited to, cellulose, starch, kaolinite, bentonite, sodium citrate, gelatin, agar, carboxymethyl cellulose, gum arabic, tragacanth, seaweed gel, glyceryl monostearate, calcium stearate, colloidal silicon dioxide, and combinations thereof.

As a form for transdermal administration, the pharmaceutical composition or medicament in accordance with the present invention can also comprise any pharmaceutically acceptable carrier that will not adversely affect the desired effects of the active ingredient (i.e., antibodies of the present invention or antigen-binding fragment thereof), such as water, mineral oil, propylene glycol, polyethylene oxide, liquid petrolatum, sorbitan monostearate, and polysorbate 60. The pharmaceutical composition or medicament can be provided by any suitable methods in any suitable form for transdermal administration, such as in the form of a patch (such as a microneedle patch), but is not limited thereby.

As for the form of injections or drips, the pharmaceutical composition or medicament can comprise one or more ingredient(s), such as an isotonic solution, a salt-buffered saline (e.g., phosphate-buffered saline or citrate-buffered saline), a hydrotropic agent, an emulsifier, a 5% sugar solution, and other carriers to provide the pharmaceutical composition or medicament as an intravenous infusion, an emulsified intravenous infusion, a powder for injection, a suspension for injection or a powder suspension for injection. Alternatively, the pharmaceutical composition or medicament can be prepared as a pre-injection solid. The desired injection is provided by dissolving the pre-injection solid in other solutions or suspensions or emulsifying it before being administered to a subject in need.

As a form for transmucosal administration, the pharmaceutical composition or medicament can comprise one or more ingredient(s), such as a penetrant, a surfactant, a viscosity regulator, a pH-adjusting agent, a preservative, a stabilizer, an osmo-regulator, and other carriers to provide the pharmaceutical composition or medicament as eye drops, an ointment, orally disintegrating tablets, a nasal spray, nasal drops, or a suppository.

Optionally, the pharmaceutical composition or medicament in accordance with the present invention can also comprise a suitable amount of additives, such as a toner or a colorant for enhancing the visual perception of the pharmaceutical composition or medicament, and/or a buffer, a conservative, a preservative, an antibacterial agent, or an antifungal agent for improving the stability and storability of the pharmaceutical composition or medicament.

The pharmaceutical composition or medicament provided in accordance with the present invention can optionally further comprise one or more other active ingredient(s) (such as PD-1 antagonist, PD-L1 antagonist, CTLA-4 antagonist, EGFR inhibitor, and A2AR inhibitor) to further enhance the effects of the pharmaceutical composition or medicament or to increase the application flexibility and application adaptability of preparation thus provided, as long as the other active ingredients do not adversely affect the desired effects of the active ingredient of the present invention (i.e., antibodies of the present invention or antigen-binding fragment thereof).

The present invention also provides a method of inhibiting CD73, comprising administering a subject in need thereof an effective amount of the antibody or antigen-binding fragment thereof as described above, wherein the term “a subject in need thereof” refers to a subject having weak immune system and/or suffering from cancer, especially the cancer with high expression of CD73. Preferably, the aforementioned cancer is selected from the group consisting of breast cancer, gastric cancer, colorectal cancer, gallbladder cancer, prostate cancer, ovarian carcinoma, chronic or acute lymphocytic leukemia, bladder cancer, brain tumor, kidney carcinoma, head and neck squamous cell carcinoma, glioblastoma, esophageal cancer, pancreatic cancer, renal carcinoma, oral cancer, lung cancer, colon adenocarcinoma, melanoma, and lymphoma. In the method in accordance with the present invention, the antibody or antigen-binding fragment thereof could be administered to the subject in need as a form of the pharmaceutical composition or medicament as described above, and the administration type, administration route, administration form, administration frequency and uses of the pharmaceutical composition and the medicament are also all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLE

### Example 1: Preparation of Anti-CD73 Antibodies

1-1. Construction of scFv Antibody Library

Recombinant Human CD73-6xHis having an amino acid sequence of SEQ ID NO: 1 was used as an antigen to immunize a mouse once a week for 6 to 9 times. After immunization, the mouse was sacrificed and the spleen and lymph nodes thereof were obtained. Total RNAs of the spleen and lymph nodes were extracted and reverse-transcribed in an RT-PCR procedure with the primers to construct antibody fragments containing VH and VL. The antibody fragments were assembled into scFv fragments in polymerase chain reaction (PCR), and a scFv library was constructed.

1-2. Preparation of scFv Phage for Bio-Panning

The scFv library provided by Example 1-1 was inoculated into a 2xYT medium containing 100 μg/ml ampicillin and 2% glucose (2YTAG) and incubated by shaking at 37° C. until the OD at 600 nm reaches 0.5. The culture was infected with a helper phage and then cultured without shaking in a 37° C. water bath for 30 min. The cells in the culture were collected and suspended in a 2xYT medium containing 100 μg/ml ampicillin and 25 μg/ml kanamycin (2YTAK) and further incubated with shaking at 30° C. overnight. The supernatant of the culture was collected and mixed with ⅕ volume of PEG/NaCl (20% Polyethylene glycol 8000, 2.5 M NaCl) and stayed at 4° C. for at least one hour. After centrifuging, the pellet was collected and suspended in PBS and spun again to collect the supernatant.

### 1-3. Bio-Panning of ScFv Phages by Using ELISA Method

An ELISA plate (Nunc) was coated with 5 to 25 μg/100 μl of antigen per well and stayed in sodium bicarbonate buffer (pH 9.6) at 4° C. overnight. The wells were washed 3 times with PBS and blocked with 300 μl of 5% skim milk-containing PBS (MPBS) per well at 37° C. for 2 hours. After being washed 3 times with PBS, 100 μl of phages in 5% MPBS with his-tag-containing fusion protein were added and incubated at 37° C. for 90 min. After being washed 4 to 10 times with 0.05% Tween 20-containing PBS (PBST) and 4 to 10 times with PBS, the phages were eluted by adding 100 μl of 100 mM triethylamine (TEA) and reacted at 37° C. for 20 min. 100 μl of eluted phages were neutralized with 50 μl of 1 M Tris, pH 7.4. 3 mL of TG1 at an exponentially growing stage were added with the eluted phages. The cultures were incubated at 37° C. for 30 min without shaking for infection. The infected TG1 bacteria were added with 20 mL of 2x YT-AG and then incubated at 37° C. overnight.

### 1-4. Preparation of Next Round Phage

The cultures provided by Example 1-3 were spun, collected, and then suspended in 0.5 mL of 2xYT-AG, 15% glycerol. Then, 10 μl of the bacteria were added to 10 mL of 2xYT-AG and the bacteria grew with shaking at 37° C. until the OD at 600 nm reaches 0.5. 10 mL of the culture was infected with M13KO7 helper phage by adding the helper phage at a ratio of 1:20 (M13KO7 helper phage: culture) and the infected culture was incubated without shaking in a 37° C. water bath for 30 min. The cultures were spun to collect the pellet, and the pellet was suspended with 25 mL of 2xYT-AK and then cultured at 30° C. overnight. Further, 25 mL of the overnight culture was spun at 10,000 rpm for 20 min to collect the supernatant, and ⅕ volume (5 mL) PEG/NaCl was added to the supernatant to provide a mixture. The mixture was spun at 10,000 rpm for 20 min and the pellet was collected and suspended in 0.5 mL PBS.

### 1-5. Screening of Human CD73-Positive Phage Clones by ELISA

The suspensions provided by Example 1-4 were spread on a plate, and cultured to obtain individual colonies. The individual colonies thus provided were inoculated into 200 μl of 2xYT-AG 96-well plates and grew with shaking at 37° C. overnight, and then 10 μl of the culture was transferred to a second 96-well plate containing 180 μl of 2×YT-A per well for shaking at 37° C. for 2 hours. Then, 50 μl of 2xYT-A with 2.4×10¹⁰ pfu/mL M13KO7 helper phage was added to each well of the second plate to provide a mixture. The mixture was shaken at 37° C. for 2 hours. 50 μl of 2xYT-AK₃ (the kanamycin concentration was 300 μg/mL) was added to the mixture, and then grew with shaking at 30° C. overnight. The culture was added with 50 μl MPBS to provide a phage mixture, and 100 μl of the phage mixture was taken for phage ELISA.

The ELISA plates were coated with 1 μg/mL of protein antigen per well, and then rinsed 3 times with PBS and blocked with 300 μl of 5% MPBS per well at 37° C. for 2 hours. After rinsing a further 3 times with PBS, 100 μl phage mixture as detailed above was added and incubated at 37° C. for 90 min. The phage solution was discarded and the wells were washed 6 times with PBST and 6 times with PBS, and then, an appropriate diluted HRP-anti-M13 antibody in 5% MPBS was added to provide a mixture. The mixture was incubated at 37° C. for 60 min, and then washed 6 times with PBST. The wells were developed with substrate solution (TMB) and the reactions were stopped by adding 100 μl of 1 M Hydrochloric acid. The color turned yellow, and the OD at 650 nm and at 450 nm was assayed.

After screening, a total of 317 clones were identified with different CDR sequences.

### 1-6. The Monoclonal Phage Preparation

The 317 clones provided by Example 1-5 were subjected to the procedure as follows. The bacteria were cultured at 37° C. overnight. Thereafter, 100 μl bacteria were added with 2xYT-AG, and the bacteria grew with shaking at 37° C. until the OD at 600 nm reaches 0.5. 10 mL of the culture was infected with M13KO7 helper phage by adding the helper phage at a ratio of 1:20 (M13KO7 helper phage: culture), and the infected culture was incubated without shaking in a 37° C. water bath for 30 min. The cultures were spun to collect the pellet, and the pellet was suspended with 25 mL of 2xYT-AK and then cultured at 30° C. overnight. Further, 25 mL of the overnight culture was spun at 10,000 rpm for 20 min to collect the supernatant, and ⅕ volume (5 ml) PEG/NaCl was added to the supernatant to provide a mixture. The mixture was spun at 10,000 rpm for 20 min, and the pellet was collected and suspended in 0.5 mL PBS.

### 1-7. Screening CD73 Phage Binding to KLM-1 Cells

To screen the phage clones that can bind to CD73 expressing cells, KLM-1 cells were mixed with anti-CD73 phages provided by Example 1-6 (10¹¹ virions/tube, 100 μl). After incubation at 4° C. for 1 hour, the cells were washed and centrifuged, and were reacted with anti-M13-FITC (1:500) to each tube. After washing and centrifuging, adding 0.3 mL of Flow Cytometry Staining Buffer to each tube. Mixing gently and analyzing by BD FACSVerse™ Flow Cytometer.

After being analyzed by flow cytometry, 27 phage clones were shown to have the function of binding to the CD73 expressing cells, KLM-1.

Then, as described in Example 1-8 as follows, a full-length antibody was expressed by further constructing the VH and VL chains of these CD73 binding phage clones into a full-length antibody expression vector.

### 1-8. Expression of Full-Length Antibodies

The genes encoding anti-human CD73 antibodies were constructed by inserting the VH and VL chains, which come from the scFv phage clones provided by Example 1-7, into an expression vector containing CH and CL chain, respectively. Free-style 293 cells were transfected with the constructed vector. The antibodies were purified by using Protein A Sepharose Fast Flow (GE Healthcare, 17-1279-02). After purification, the antibodies were quantified by measuring at OD 280 nm and checked by reducing and non-reducing PAGE.

### 1-9. Binding Affinity Assay by Using ELISA

An ELISA plate was coated with 100 μl per well of human CD73 at 4° C. overnight, and then rinsed 3 times with PBS and blocked with 300 μl per well of 5% MPBS at 37° C. for 2 hours. The wells were rinsed 3 times with PBS, and 100 μl of anti-human CD73 antibody (with 2-fold serial dilutions) was added thereinto and incubated at 37° C. for 90 min. The test solution was discarded and washed 3 times with PBS. Appropriately diluted antibodies provided by Example 1-8 in 5% MPBS (1:10000) were added to the wells, and the wells were incubated at 37° C. for 60 min, and then the wells were washed 3 times with PBS. The wells were developed with 100 μl of substrate solution TMB and the reactions were terminated by adding 50 μl of 1 M sulfuric acid sulphuric acid. The color turned yellow and the OD at 650 nm and at 450 nm were assayed.

The binding affinity value (K_{D}) of antibodies provided by Example 1-8 was calculated by the equation of one site binding (hyperbola) of the GraphPad Prism software. The antibody whose K_{D} was smaller than 10⁻⁹M was chosen for the next functional assay of the cellular CD73 activity inhibition.

### Example 2: Selecting the Antibody with a Better Effect of Inhibiting CD73 Function

To investigate the inhibitory effect of selected antibodies on CD73 activity in CD73 highly expressing cells, the following experiments were conducted by using the AMP consumption assay as the method from Hay et al (Oncoimmunology 2016, 5, 1208875). It has been known that AMP can inhibit the ability of ATP in emitting luminescence. Thus, the consumption of AMP can be determined by measuring the luminescence level. Furthermore, AMP can be converted into adenosine by the membrane protein CD73, thus, the lower luminenscence level, the lower AMP consumption, and the higher the inhibitory effect of the antibody on the CD73. MDA-MB-231 cells were previously seeded into a 96-well plate (2.5×10³ cells/well) for 16 hr, and then cells in each well were treated with the following conditions: cells cultured with L-15 medium containing 100 μM AMP and antibody (8 concentrations with serial dilution) in each group, including isotype IgG, Oleclumab, and selected antibodies, at 37° C. incubator for 24 hours, supernatants of each group were collected into round-bottom 96-well plate, and then ATP was added into in a final concentration of 100 μM. There are several control treatments: the “Cell+ATP” group means that cells were only cultured with L-15 medium without antibody and AMP, and then ATP was added thereinto; the “Cell+AMP+ATP” means that cells were cultured with L-15 medium containing 100 μM AMP, and then ATP was added thereinto; the “ATP only” means L-15 medium without cells incubation, and then ATP was added thereinto; the “AMP+ATP” means medium containing 100 μM AMP without cells incubation, and then ATP was added thereinto.

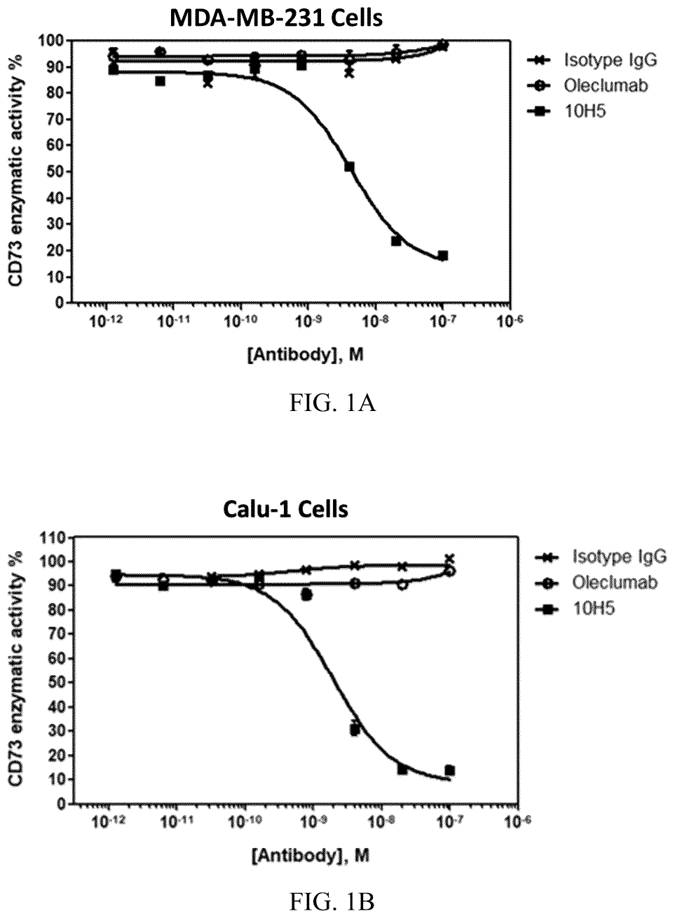

After the above procedure was completed, the medium of each group was mixed with Celltiter-glo reagent at a volume ratio of 1:1 and incubated in the dark for 10 min. Luminometer was used for measuring the luminescence of each group. The CD73 enzymatic activity (i.e., the consumption % of AMP) of each group can be calculated by the below Formula 1, and the results are shown in FIG. 1.

$$consumption \% of AMP = \frac{luminescenceofexperimentalgroup - lu min escenceof ATP + AMP group}{lu min escenceof ATPonlygroup - lu min escenceof ATP + AMP group} \times 100 \% \qquad Formula 1 :$$

2-1. Identify the Antibody which has a Better Inhibiting CD73 Activity Function

After the antibodies selected by both cell-based (flow assay) and antigen (ELISA) binding affinity assays, the next-step were checking the inhibitory effect of these antibodies on CD73 activity by using the AMP consumption assay as described above. All the selected antibodies have no significant better effect on inhibiting CD73 activity than the reference antibody, Oleclumab. But only the 10H5 have the significantly inhibiting CD73 superior than Oleclumab. As shown in FIG. 1A, after 24 hours of culturing MDA-MB-231 cells and 100 μM AMP with reference antibody Oleclumab, the AMP was almost consumed. However, the “10H5” group can significantly inhibit the consumption of AMP, and the consumption of AMP was even reduced to be the lowest level (<20%) when the concentration of 10H5 was higher than 10 nM. These results indicate that, as compared to the Oleclumab, the 10H5 has a superior and better inhibitory (in 24 hours) effect on CD73 activity on MDA-MB-231 cancer cells.

2-2. Confirm the 10H5 with a Novel CD73 Inhibition Function by NSCLC Calu-1 Cells

To confirm the 10H5 with a superior inhibitory effect of CD73, the CD73 highly expressing NSCLC Calu-1 was used for testing whether it also works as in MDA-MB-231.

NSCLC Calu-1 cells were seeded into a 96-well plate (500 cells/well), and cultured with McCoy's 5a medium containing 100 μM AMP and antibodies, including isotype IgG, Oleclumab and 10H5 (serial dilution with 8 concentrations), at 37° C., 5% CO₂ incubator for 24 hours, and then 100 μM ATP was added thereinto. Other control groups and following procedure are described as above, and the results are shown in FIG. 1B.

As shown in FIG. 1B, after 24 hours of culturing, the “Oleclumab” group, the AMP was also almost consumed. However, the “10H5” group can still significantly inhibit the consumption of AMP, and the consumption of AMP was reduced to be even lowest (10%) when the concentration of 10H5 was higher than 10 nM. These results indicate that, as compared to the Oleclumab, the antibody of the present invention, 10H5, has a better inhibitory (in 24 hours) effect on CD73 activity on Calu-1 cancer cells.

### Example 3: Humanization of Antibody

### 3-1. CDR Grafting

Since the 10H5 antibody was found to have a novel and special inhibitory effect on CD73 activity, we humanized the 10H5 antibody for further development as a therapeutic antibody. As described above, the variable region of the 10H5 antibody was derived from the mouse scFv sequence. To make a humanized 10H5 antibody, the framework of 10H5 antibody was replaced with a human framework selected from the IMGT database (www.imgt.org/) (heavy chain: IGHV1-46*01F; light chain: IGKV1-12*01). And, to replace the whole framework of the 10H5 antibody with a human framework, 21 amino acids in the heavy chain and 26 amino acids in the light chain were mutated. Thus, the variation of the heavy chain and the light chain was 25.6% (21 mutations/82 amino acids in the heavy chain) and 34.2% (26 mutations/76 amino acids in the light chain), respectively.

### 3-2. Back Mutation

Because the framework of humanized antibody may directly or indirectly affect the binding affinity to antigen or the inhibitory effect on CD73 activity. Therefore, some specific amino acid residues on the human framework would need to be mutated back to that of the mouse framework.

### 3-3. Inhibitory Effect of the Humanized Antibodies on CD73 Activity

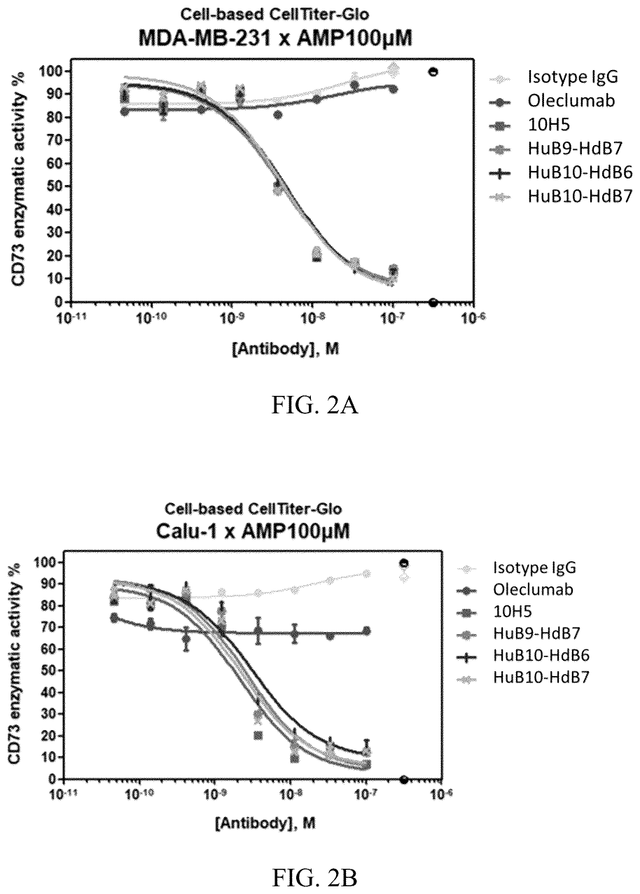

The humanized antibodies were selected by three criteria: the binding affinity, the CD73 inhibitory activity, and the minimum number of back mutations. There are three antibodies, HuB9/HdB7, HuB10/HdB6 and HuB10HdB7, which are combined by two heavy chains: HuVHB9 (HuB9) and HuVHB10 (HuB10) and two light chains: HdVLB6 (HdB6) and HdVLB7 (HdB7), that meet these three conditions. The amino acid sequences of HuB9, HuB10, HdB6, and HdB7 were shown in SEQ ID NO: 10 (HuB9), SEQ ID NO: 11 (HuB10), SEQ ID NO: 12 (HdB6), and SEQ ID NO: 13 (HdB7). To further investigate the characters of the humanized antibodies of the present invention, the abilities of HuB9/HdB7, HuB 10/HdB6 and HuB10/HdB7 on inhibiting AMP consumption (i.e., inhibiting CD73 activity) were analyzed by the AMP consumption assay as described in Examples 2. The results are shown in FIG. 2A and 2B.

As shown in FIG. 2A and 2B, HuB9/HdB7, HuB10/HdB6 and HuB10/HdB7 can provide a similar inhibitory effect on AMP consumption as 10H5 antibody on the MDA-MB231 (FIG. 2A) and Calu-1 (FIG. 2B) cells.

### Example 4: Binding Affinity of the Antibodies of the Present Invention

To investigate the characters of the antibodies of the present invention, including 10H5 and its humanization derivatives, HuB9/HdB7, HuB10/HdB6 and HuB10/HdB7, we analyzed the binding affinity of these antibodies by Biacore SPR.

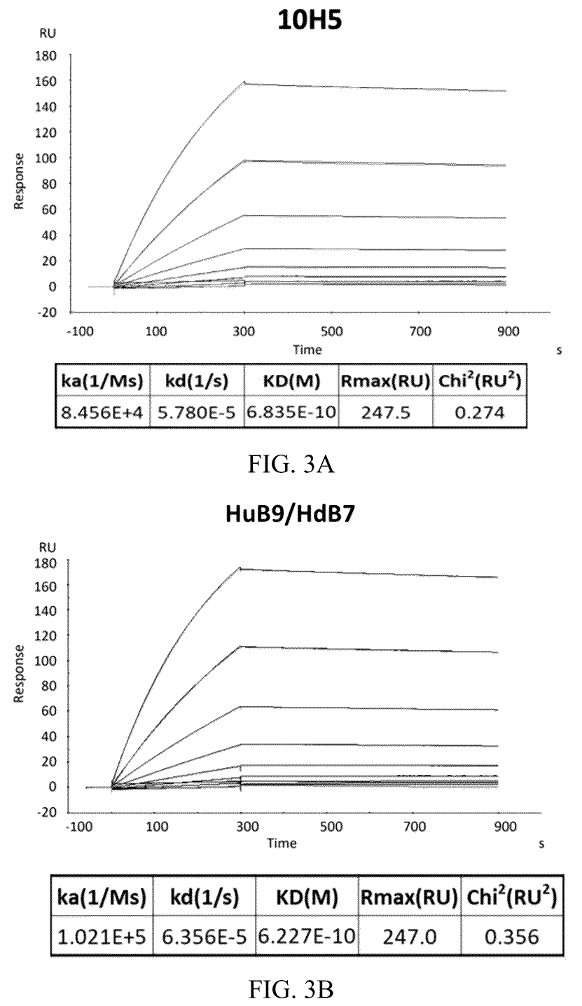

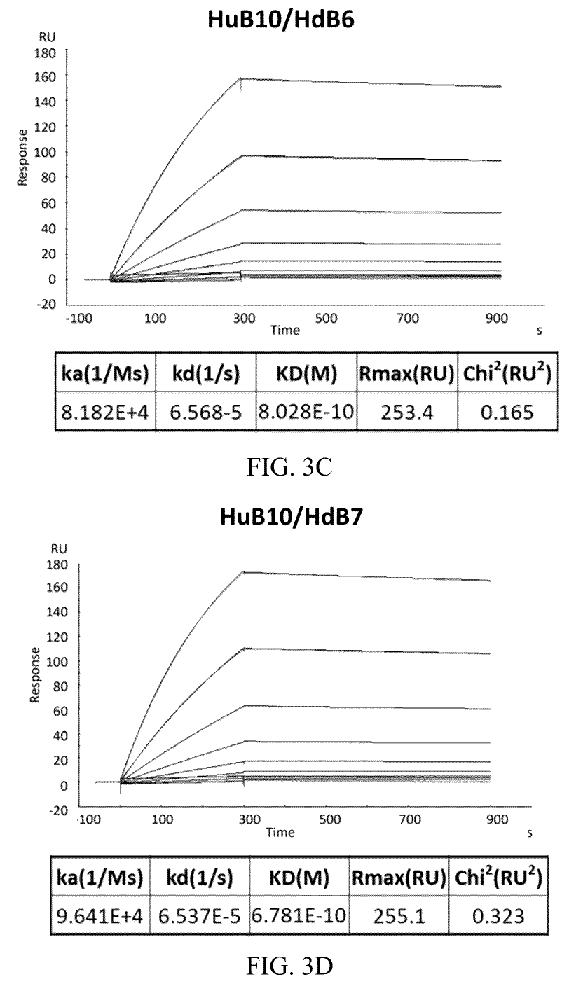

The analyzing condition was flow rate 30 μL/min, associate time 180s, dissociate time 600s; the concentrations of 10H5 antibody were 0.625 nM, 1.25 nM, 2.5 nM, 5 nM and 10 nM). The binding affinity of 10H5 and its humanization derivatives HuB9/HdB7, HuB10/HdB6 and HuB10/HdB7 are shown in FIGS. 3A, 3B, 3C and 3D, respectively.

As shown in FIG. 3A, the KD value of 10H5 antibody measure by Biacore SPR was 6.835×10⁻¹⁰M. As shown in FIGS. 3B, 3C and 3D, the KD value of HuB9/HdB7, HuB10/HdB6 and HuB10/HdB7 antibodies measured by Biacore SPR was 6.227, 8.028 and 6.781×10⁻¹⁰M, respectively. All the three humanized antibodies have a similar binding affinity to human CD73 as parental 10H5 antibody.

### Example 5: Long-Lasting Inhibitory Effect of the Antibodies of the Present Invention on CD73 Activity

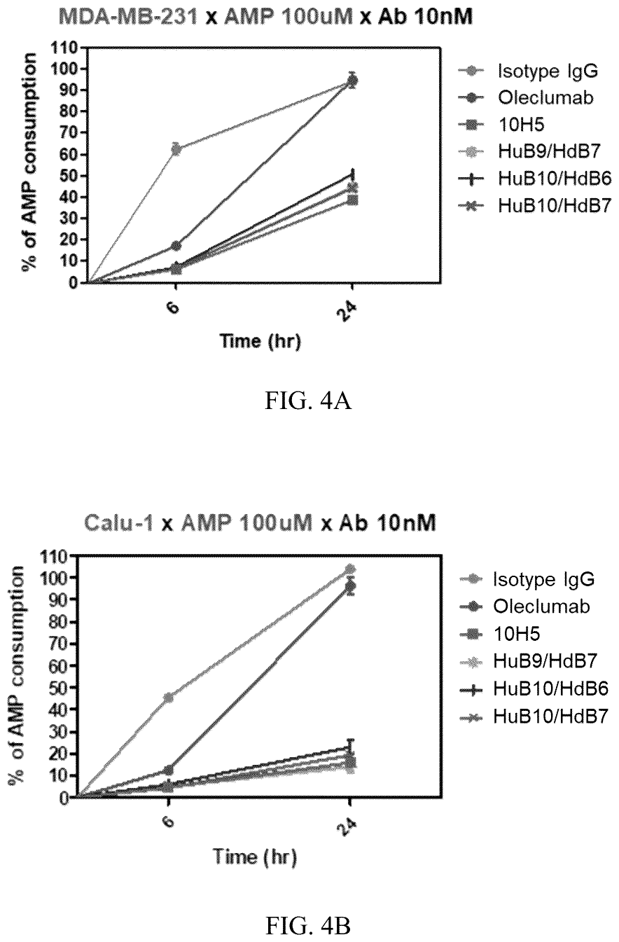

To further check the antibodies of the present invention have the more long-lasting inhibitory effect on CD73 than oleclumab, we treated the antibodies, 10H5 and its humanized derivatives with both MDA-MB-31 and Calu-1 cells. After 6 and 24-hours treatment, the inhibitory effect was analyzed by AMP consumption assay. The experimental operation method and process are the same as the Example 2, but the antibody concentration was only 10 nM, and the incubation time was 6 hr and 24 hr. The CD73 activity results were presented in FIG. 4A and FIG. 4B.

As shown in the FIG. 4A, after 6 hr of the MDA-MB-231 cells treated with AMP and antibody, the AMP consumption was obviously found in the “isotype IgG” group (about 60%). But the AMP consumption was less inhibited by all the antibodies treatment, including “Oleclumab” (less than 20%), “10H5” and its derivatives (less than 10%). However, after 24 h, the AMP of the “Oleclumab” was seen almost consumed completely (above 90%). But the “10H5” and its derivatives were found significantly less AMP consumption (less than 50%) than “Oleclumab” in the MDA-MB-231 cancer cells. As shown in the FIG. 4B, the same tendency result was present in the data of Calu-1 cells, after 6-hr, the AMP was also seen less consumption in “Oleclumab” and 10H5 series, but after 24 hrs, the AMP of the “Oleclumab” was seen almost consumed (above 90%). However, the “10H5” and its derivatives were found significantly less AMP consumption (<20%) than Oleclumab in the Calu-1 NSCLC cells. These results indicate that the antibodies of the present invention, 10H5 and its humanized derivatives, have significantly better and more long-lasting inhibitory property on CD73 activity than Oleclumab.

### Example 6: Analyzing the Inhibitory Effect of the Antibodies of the Present Invention on CD73 Activity by Measuring Extracellular AMP Concentration

The AMP consumption measured in Examples 2, 3 and 5 was inferred from the luminescence levels. To investigate the actual AMP concentration changed by the reaction of CD73 in cells and antibody, the following experiments by measuring the AMP concentration were conducted.

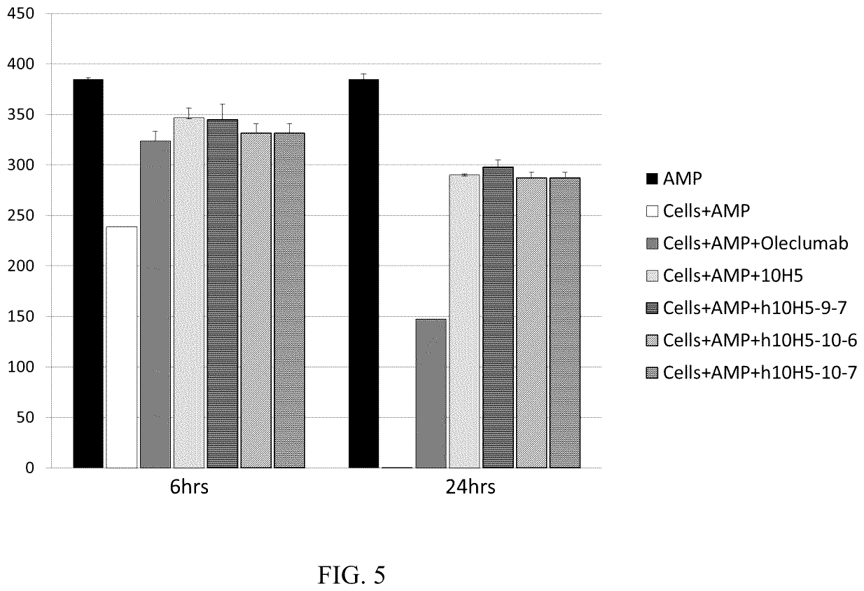

MDA-MB-231 cells were seeded into a 12-well plate (2.5×10⁴ cells/well), and cells in each well were cultured with 1.5 ml serum-free medium containing 400 μM AMP and 10 nM antibody, including Oleclumab, 10H5 or its derivatives. The following condition is at 37° C. for 6 hours or 24 hours. There are two control groups as follows: The “AMP only” group means the 1.5 ml medium containing 400 μM AMP (without cells); and the “Cell+AMP” group means that cells were cultured with 1.5 ml serum-free medium containing 400 μM AMP without any antibody. After the above procedure was completed, the AMP concentration in the supernatant was measured by LC/MS. The results are shown in FIG. 5

As shown in FIG. 5, after 6 hours of culturing, the AMP concentration of the “AMP only” group was 385 μM, but that of the “Cell+AMP” group was 239 μM, which demonstrated that the MDA-MB-231 cells can consume the AMP in 6 hours. However, the “Cell+AMP+Oleclumab” group (i.e., adding Oleclumab) can maintain an AMP concentration of 324 μM, and the “Cell+AMP+10H5” group (i.e., adding the 10H5 antibody) can maintain an AMP concentration of 347 μM. The humanized antibodies HuB9/HdB7, HuB10/HdB6 and HuB 10/HdB7 retained the AMP with almost the same as 10H5, which was 345, 336 and 331 μM, respectively. These results indicate that Oleclumab, 10H5 and its derivatives slightly and equally inhibit the AMP consumption of MDA-MB-231 cells in 6 hours. After 24 hours of culturing, the AMP concentration of the “AMP only” group still maintained at 385 μM, but that of the “Cell+AMP” group was only 0.5 μM, which demonstrated that the MDA-MB-231 cells can consume the entire AMP after 24 hours. The “Cell+AMP+Oleclumab” group (i.e., adding Oleclumab) can only retain the AMP with the AMP concentration of 147 μM. However, the “Cell+AMP+10H5” group (i.e., adding the 10H5 antibody of the present invention) can maintain an AMP concentration of 290 μM. The treatment of humanized antibodies HuB9/HdB7, HuB10/HdB6 and HuB10/HdB7 after 24hrs also retained the same AMP concentrations as 10H5, which was 298, 286 and 287 μM, respectively. These results indicate that, compared to the Oleclumab, antibodies of the present invention (e.g., 10H5) have significantly better and more long-lasting inhibitory effect on CD73 activity of MDA-MB-231 cells.

### Example 7: Activation Effect of the Antibodies of the Present Invention on T-Cells

It is known that CD3/CD28 dynabeads can induce the cell division of CD4⁺T-cells. Furthermore, the cell division situation can be known by dying the CD4⁺T-cells with CFSE and analyzing the fluorescence level of the dyed CD4⁺T-cells (the more the cell division occurred, the weaker the fluorescence being emitted, which makes the peak in the flow cytometry chart be left-shifted). To investigate whether the 10H5 antibody of the present invention can activate the immune cells (such as CD4⁺T-cell), the following experiments were conducted.

Firstly, 20 mL of human blood was drawn from a donor, and mixed with 20 ml PBS. The blood mixture was slightly added into 50 ml tube containing 15 ml Lymphoprep (STEMCELL Tech.). Centrifuged at 2000 rpm for 30 min without slow down. The thin layer in white color located between the serum and Lymphoprep was PBMC. PBMC was collected and put into a new 50 ml tube. The tube was washed with PBS 3 times, and then CD4⁺T-cells in the PBMC were isolated by using a CD4 isolation kit and calculated.

An appropriate number of CD4⁺T-cells were collected and centrifuged. Thereafter, 1 μM CFSE was used to stain CD4⁺T-cells to evenly make the cell concentration be 1×10⁶/ml. The cells were then cultured at 37° C. incubator for 20 min and washed with medium 2 times. Thereafter, an appropriate number of cells were collected and centrifuged. The cells were then re-suspended to make the cell concentration to be about 1×10⁶/ml. The cells were cultured in medium containing 30IU/mL IL-2, CD3/CD28 dynabeads that equivalent to the numbers of cells, and 10 nM hIgG, Oleclumab, 10H5 or its humanized derivatives, HuB9/HdB7, HuB10/HdB6 or HuB 10/HdB7 were added, and cells were cultured in a 37° C. incubator for 60 min and then seeded into a 96-well plate (100 μl/well) with additional 400 μM AMP and cultured in a 37° C. incubator for 3 to 4 days.

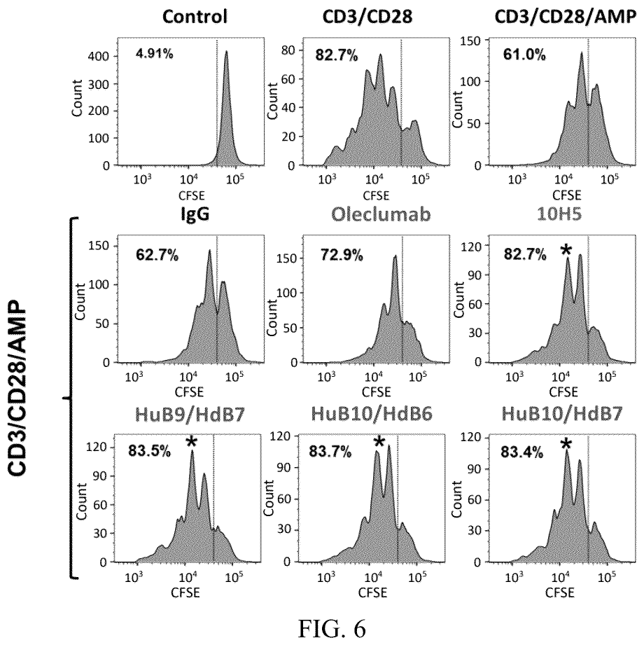

The “Control” group means without any CD3/CD28 dynabeads and IL-2 or antibody or AMP were added. The “CD3/CD28” group means that cells were activated by IL-2 and CD3/CD28 dynabeads, but not any antibody or AMP were added after that. The “CD3/CD28/AMP” group means that cells were activated by IL-2 and CD3/CD28 dynabeads, and then cells were cultured for 60 min and seeded into 96 well plated, then added with additional 400 μM AMP and cultured in a 37° C. incubator for 3 to 4 days; After the above procedure was completed, the cells of each group were collected and analyzed with flow cytometry. The results are shown in FIG. 6.

As shown in FIG. 6, after 3 days of culturing, the fluorescence in the “CD3/CD28” group had a left-shift of 82.7%, which was much more than that of the “control” group (4.91%). However, the “CD3/CD28/AMP” group had a left-shift of only 61.0% due to the inhibitory effect of adenosine (which was converted from AMP by the CD73 on the CD4+ T-cells) on cell division. It was also seen when 10 nM of IgG was added before the addition of 400 μM AMP, the level (62.7%) of the left-shift was almost the same as the “CD3/CD28/AMP” group. But, when 10 nM of Oleclumab was added before the addition of 400 μM AMP, it can protect AMP from being converted to adenosine, and thus reverse the left-shift level from 62.7% to 72.9%. Furthermore, the effect of 10H5 with the same dose of 10 nM, the left-shift level was seen from 62.7% to 82.7%, indicating the antibody 10H5 has more efficacy on protecting AMP being converted to adenosine and its ability in reversing the left-shift level is even better than Oleclumab. These results indicate that the antibodies of the present invention (e.g., 10H5) are effective in activating immune cells.

The activating effect of the humanized antibodies of the present invention on CD4 T-cells was also measured. HuB9/HdB7, HuB10/HdB6 and HuB10/HdB7 can also prevent the conversion from AMP to adenosine, and thus reverse the left-shift level (83.5%, 83.7% and 83.4%, respectively). The aforesaid effect was similar to that of the 10H5 antibody. These results indicate that the humanized antibodies of the present invention can provide an activation effect on T-cells.

### Example 8: Inhibitory Effect of the Antibodies of the Present Invention on the Tumor Growth in Mouse Model

It is known that a nude mouse is a naturally mutated mouse that lacks T-cells. Thus, in the following experiments, the nude mouse was used as the model to test whether the antibodies of the present invention can inhibit tumor growth in vivo.

Researches have shown that the expression of CD73 will affect the tumorigenicity of MDA-MB-231 tumor cells (Clin Exp Metastasis (2007) 24:439-448; and Cancer Sci. 2010, vol. 101, 2561-2569). Therefore, MDA-MB-231 xenograft is the major animal model for testing the in vivo effects of an anti-CD73 antibody. In view of the above fact, the following experiment was carried to evaluate the in vivo activities of the antibodies of the present invention.

### 8-1. The Inhibitory Effect of the Antibodies of the Present Invention Against Tumor Growth in Mouse Model

To investigate the inhibition of tumor growth by the 10H5 and its humanized derivatives, HuB9/HdB7, HuB10/HdB6 and HuB10/HdB7, Twenty-five Balb-C/Nu mice were used to build a xenograft model and then divided into 5 groups (5 mice/per group). MDA-MB-231 cells were injected into the mice, and when the tumor had grown to have a size of 170 mm³, the mice were injected with 10H5 and the three humanized antibodies, HuB9/HdB7, HuB10/HdB6 and HuB10/HdB7 30 mpk (mg/kg), twice a week (8 times in total).

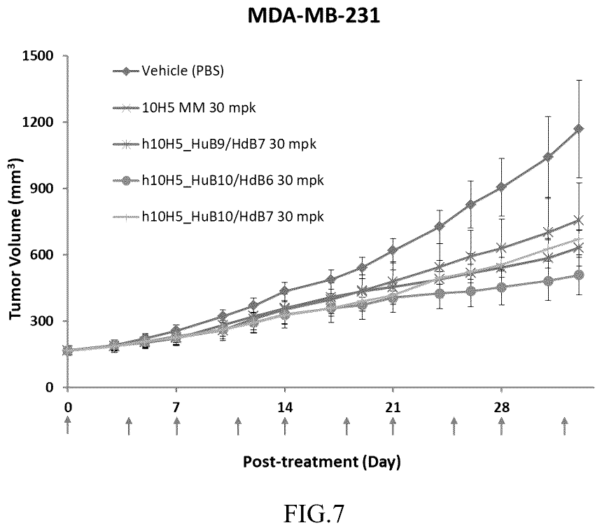

The tumor size of mice in each group was observed 2 times a week. The results are shown in FIG. 7 and the following Table 1.

TABLE 1

| Group | TGI (%) |
|---|---|
| Vehicle (PBS) | 0.0 |
| 10H5 MM | 53.6 |
| HuB9/HdB7 | 41.1 |
| HuB10/HdB6 | 65.8 |
| HuB10/HdB7 | 49.5 |

As shown in FIG. 7 and Table 1, as compared to the “Vehicle (PBS)” group, the growth of tumors in the 10H5, HuB9/HdB7, HuB10/HdB6 and HuB10/HdB7 treatments were all inhibited. The ratio of tumor growth inhibition (TGI) of the 10H5, HuB9/HdB7, HuB10/HdB6 and HuB10/HdB7 was 53.6%, 41.1%, 65.8%, and 49.5%, respectively. These results indicate that the 10H5 and its humanized derivatives all can inhibit the MDA-MB-231 tumor growth in Balb-C/Nu mice model.

### 8-2. The Inhibitory Effect of the Antibodies of the Present Invention Against the CD73 Activity in Tumor Tissues

In order to further analyze that 10H5 antibodies actually have the ability to inhibit CD73 activity in tumor tissues, MDA-MB-231 cells formed tumors in Balb-C/Nu first, then different doses (3, 10 and 30 mpk) of 10H5 were injected into the tail vein of mice. The blood was collected 24 hours later, then sacrificed the animals to remove the tumors and to evaluate the CD73 activity in each tumor tissues. We analyzed the concentration of antibody in blood by MSD assays. The concentrations of 10H5 in the blood were presented as Table 2. The concentrations of 10H5 were exactly increased following the dose of the antibody administered (Table 2).

TABLE 2

| | Concentration (μg/ml) of |
|---|---|
| Group | antibody in blood after 24 h |
| 10H5 3 mpk | 14.4 ± 4.02 |
| 10H5 10 mpk | 36.3 ± 19.2 |
| 10H5 30 mpk | 58.0 ± 5.27 |

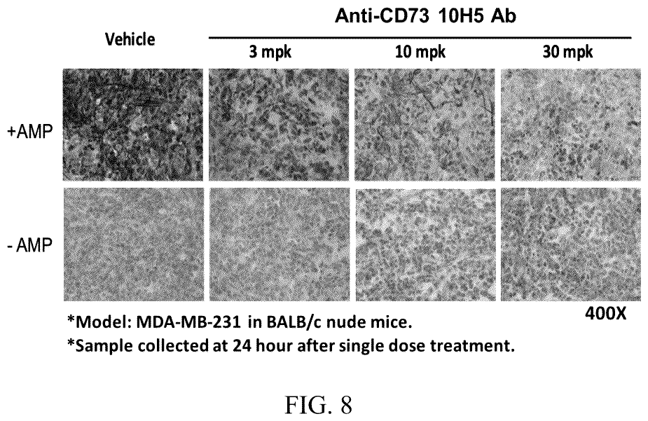

After the tumor tissue was cryosectioned, the tumor tissue was stained in situ by using the method by Mireia et al. (in situ CD73 activity assay: In Situ Identification of Ectoenzymes Involved in the Hydrolysis of Extracellular Nucleotides DOI: dx.doi.org/10.5772/intechopen.84495). The lighter of the staining, the more CD73 activity is inhibited by the antibody. As shown in FIG. 8, the tissue of tumors with vehicle treatment, the result of the staining is deep brown. However, the staining of tumors was gradually turning to light brown following the increase doses of 10H5 treatment. The phenomenon of dose-dependent in situ CD73 inhibition were also observed in humanized antibodies.

### Example 9: Epitope Mapping

To investigate the epitope of the antibody of the present invention, the following experiments were conducted.

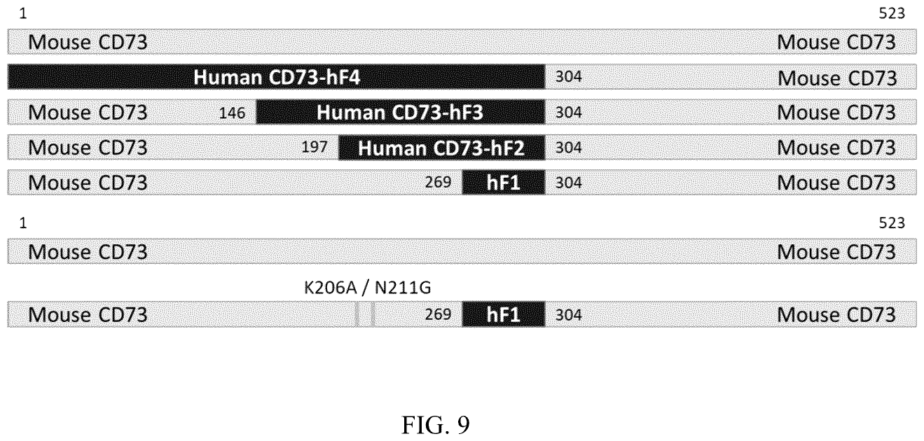

Five mouse-human domain swapping proteins were constructed. As shown in FIG. 9, the five proteins have sequences as follows:
1. mCD73hF4: mouse CD73 with 1-304 swapped to the amino acid sequence of human CD73;
2. mCD73hF3: mouse CD73 with 146-304 swapped to the amino acid sequence of human CD73;
3. mCD73hF2: mouse CD73 with 197-304 swapped to the amino acid sequence of human CD73;
4. mCD73hF1: mouse CD73 with 269-304 swapped to the amino acid sequence of human CD73; and
5. mCD73hF1-K206A-N211G: not only mouse CD73 with 269-304 swapped to the amino acid sequence of human CD73, but also the lysine residue at position 206 and asparagine residue at position 211 were substituted with alanine and glycine, respectively

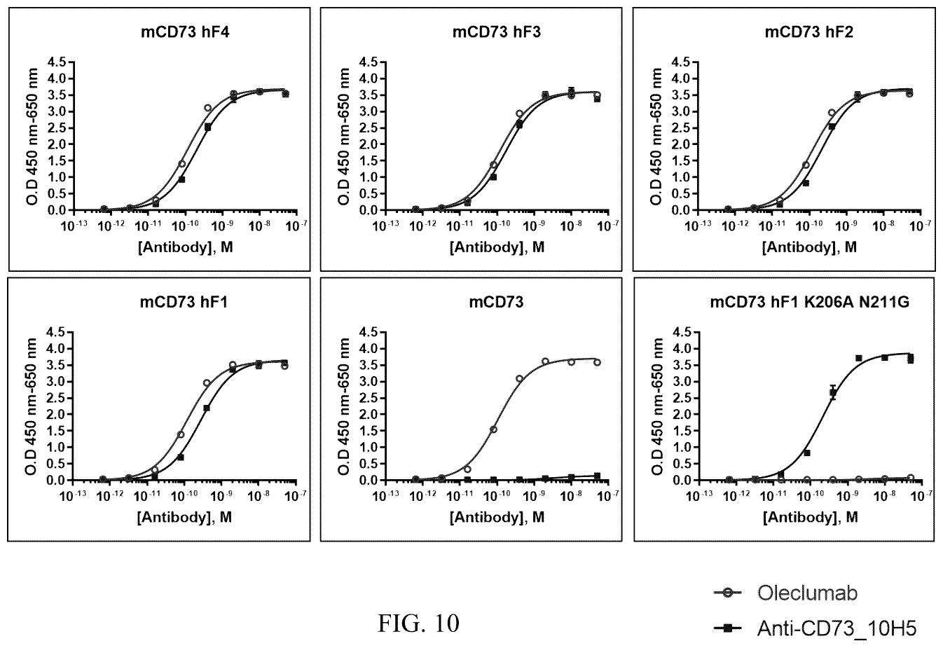

The above five mouse-human domain swapping proteins and mouse CD73 were individually cloned into F293 cells to express. By using ELISA assay, those proteins were then be analyzed the binding affinity of anti-CD73 antibodies (e.g., Oleclumab and 10H5). The results are shown in FIG. 10.

As shown in FIG. 10, Oleclumab can bind to mouse CD73 and mCD73hF1 to mCD73hF4, while cannot bind to mCD73hF1-K206A-N211G (the loss of this mutant binding to MEDI9447 has been disclosed in patent US9938356B2). That is, Oleclumab can binds to both mouse CD73 and human CD73 on the lysine residue at position 206 and asparagine residue at position 211. On the other hand, 10H5 can bind to mCD73hF1 to mCD73hF4 and mCD73hF1-K206A-N211G, while cannot bind to mouse CD73. These results indicate that 10H5 has species specificity and binds to the CD73 on the amino acids at positions 269-304. It also shows that 10H5 is different from Oleclumab by the binding site at 269 to 304 which is different from the K206 and N211 recognized by Oleclumab.

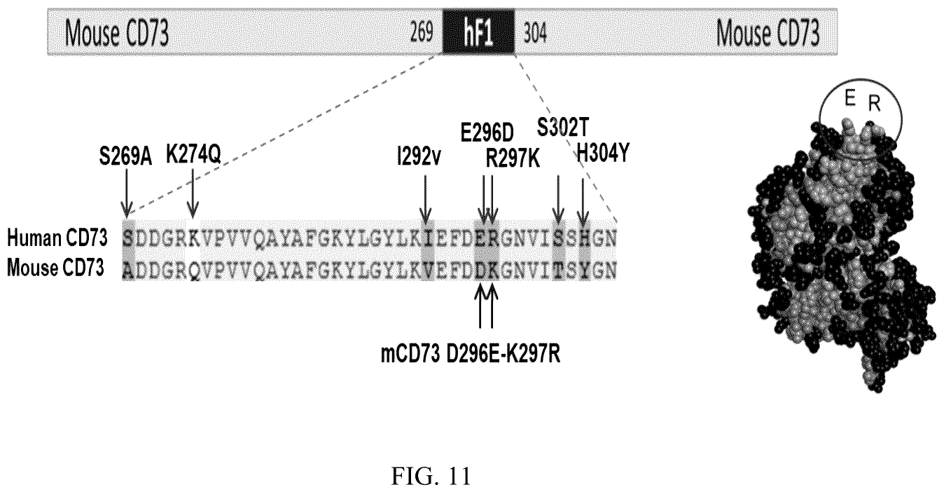

To further clarify the position 269 to 304 of CD73 being bound by 10H5 antibody, the amino acid residue at positions 269, 274, 292, 296, 297, 302, and 304, which are different between human CD73 and mouse CD73, were mutated individually to check the binding epitope of the 10H5 antibody. Thus, mCD73hF1 was made further mutations on the above positions to construct the following eight proteins: mCD73hF1-S269A, mCD73hF1-K274Q, mCD73hF1-1292V, mCD73hF1-E296D, mCD73hF1-R297K, mCD73hF1-S302T, mCD73hF1-H304Y and mCD73hF1-E296D-R297K. The positions of the mutation sites are shown in FIG. 11.

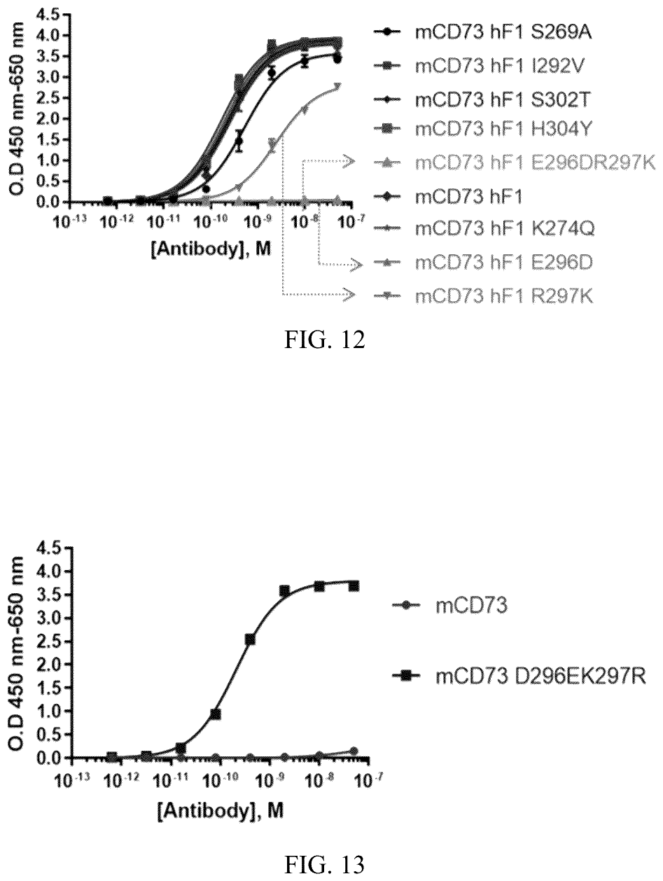

The above eight proteins and mCD73hF1 were respectively cloned into F293 cells to express, and then the binding affinity between 10H5 antibody and those proteins was analyzed by using ELISA. The results are shown in FIG. 12.

As shown in FIG. 12 10H5 can completely binds to mCD73hF1, mCD73hF1-S269A, mCD73hF1-K274Q, mCD73hF1-1292V, mCD73hF1-S302T, and mCD73hF1-H304Y, but the binding affinity to mCD73hF1-R297K (KD=2.49×10⁻⁹) was only about one tenth of that to mCD73hF1 (KD=2.54×10⁻¹⁰). When the glutamic acid residue at position 296 was mutated (i.e., mCD73hF1-E296D and mCD73hF1-E296D-R297K), the binding affinity between 10H5 antibody and CD73 would disappear.

These results indicate that the antibodies of the present invention (e.g., 10H5) bind to the CD73 dependent on glutamic acid residue at position 296 and the arginine residue at position 297.

Furthermore, two mutations were introduced into mouse CD73, wherein the aspartate residue at position 296 was substituted with glutamic acid residue and the lysine residue at position 297 was substituted with arginine residue (hereinafter referred to as “mouse CD73-D296E-K297R”) (FIG. 13). The binding affinity of 10H5 between mouse CD73 and mouse CD73-D296EK297R was analyzed by using ELISA. The results are shown in FIG. 13

As shown in FIG. 13, 10H5 antibody cannot bind to mouse CD73, while can bind to mouse CD73-D296EK297R. These results indicate again that the antibodies of the present invention (e.g., 10H5) bind to the CD73 dependent on glutamic acid residue at position 296 and the arginine residue at position 297.

### Example 10: Mapping the Binding Positions of Antibodies of the Present Invention to CD73

10-1. 3D Structure Model Simulation of the Interactions Between CD73 and 10H5 scFv Antibody

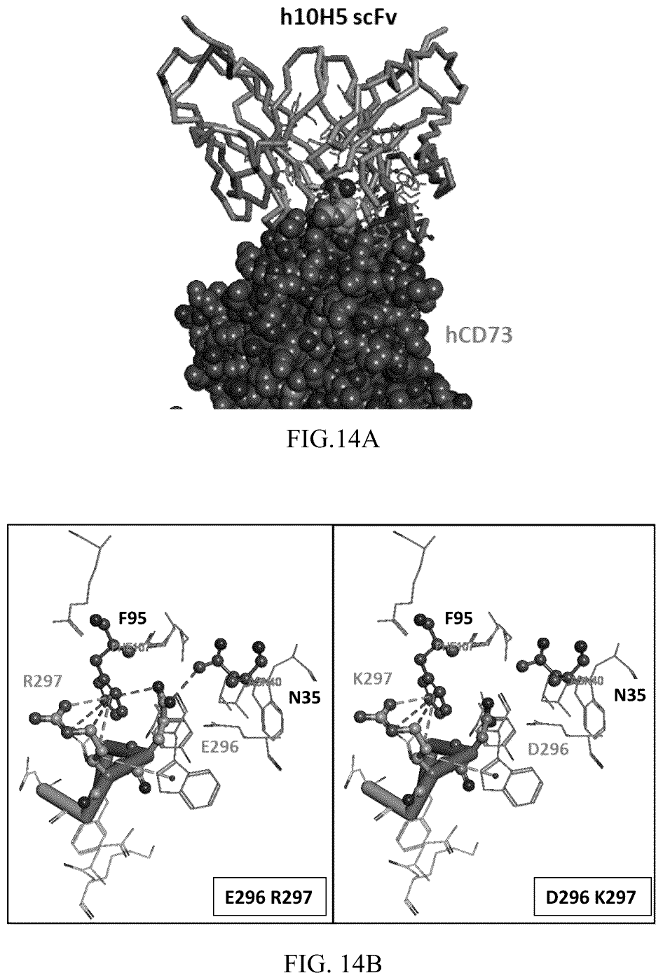

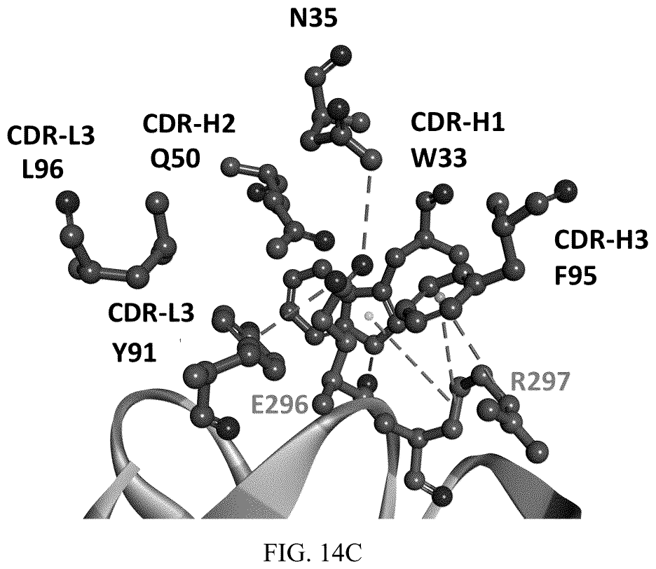

To identify the amino acids on the 10H5 antibody that involves in its interaction with E296/R297 of human CD73 antigen, 3D structure model simulation and logical intelligence analysis were used (FIG. 14A).

As shown in FIG. 14B, 10H5 scFv antibody binds to the CD73 on glutamic acid residue at position 296 and the arginine residue at position 297, and if the residues at these two positions were respectively mutated to be aspartic acid and lysine, the binding between 10H5 scFv antibody and CD73 disappeared. According to FIGS. 14A and 14B, E296/R297 of human CD73 might be surrounded by four loops of 10H5 antibody (i.e., CDR-H1, CDR-H2, CDR-H3, and CDR-L3), and potential six amino acids (W33 of CDR-H1, N35 of CDR-H1, Q50 of CDR-H2, F95 of CDR-H3, Y91 of CDR-L3 and L96 of CDR-L3) on the antibody that can generate hydrogen bond, hydrophobic effect and ion-ion interaction with CD73 (FIG. 14C).

### 10-2. Binding Positions Mapping

To investigate the binding positions of the antibody of the present invention and confirm the prediction of the 3D model as described above, the following experiments were conducted.

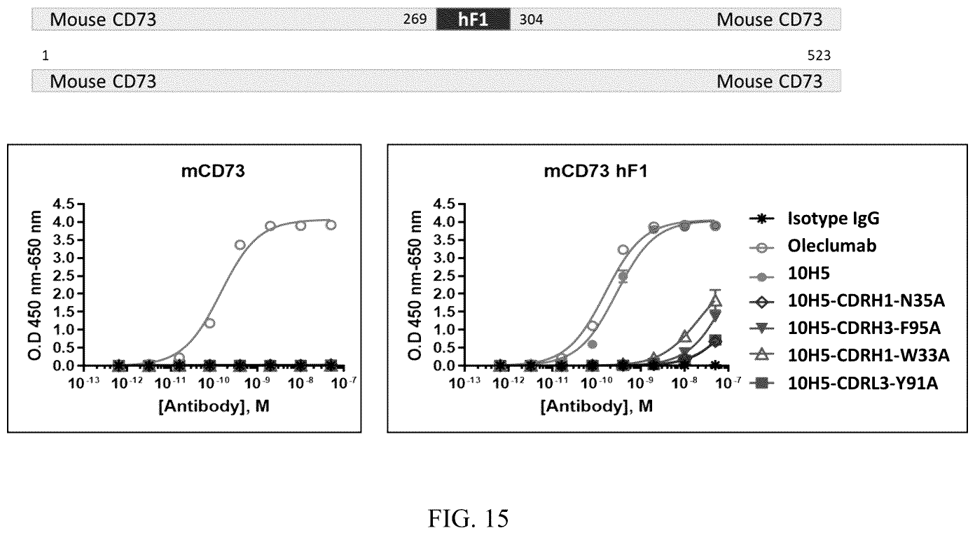

To prove the prediction of the 3D model as described above is true, the four mutated antibodies were constructed and expressed, including 10H5-CDRHIN35A, 10H5-CDRH3F95A, 10H5-CDRH1W33A and 10H5-CDRL3Y91A, to examine whether these antibodies will be loss of function. The binding affinities of the above four mutated antibodies, 10H5 antibody, Oleclumab, and IgG to mouse CD73 and mouse CD73 hF1 were respectively analyzed by using ELISA assay. The results are shown in FIG. 15.

As shown in FIG. 15, Oleclumab can bind to mouse CD73 and mCD73hF1, while 10H5 can only bind to mCD73hF1. On the other hand, 10H5-CDRHIN35A, 10H5-CDRH3F95A, 10H5-CDRH1W33A, and 10H5-CDRL3Y91A cannot bind to both the mouse CD73 and mCD73hF1. These results indicate that the W33 of CDR-H1, N35 of CDR-H1, F95 of CDR-H3, and Y91 of CDR-L3 are indeed involved in the binding of the antibody of the present invention (e.g., 10H5) to human CD73. That is, the 3D structure model established in Example 15-1 is correct. W33 of CDR-H1, N35 of CDR-H1, Q50 of CDR-H2, F95 of CDR-H3, Y91 of CDR-L3, and L96 of CDR-L3 are the key binding positions on 10H5 antibody.

10-3. Screening and Selecting Antibodies that have Similar Epitope as 10H5

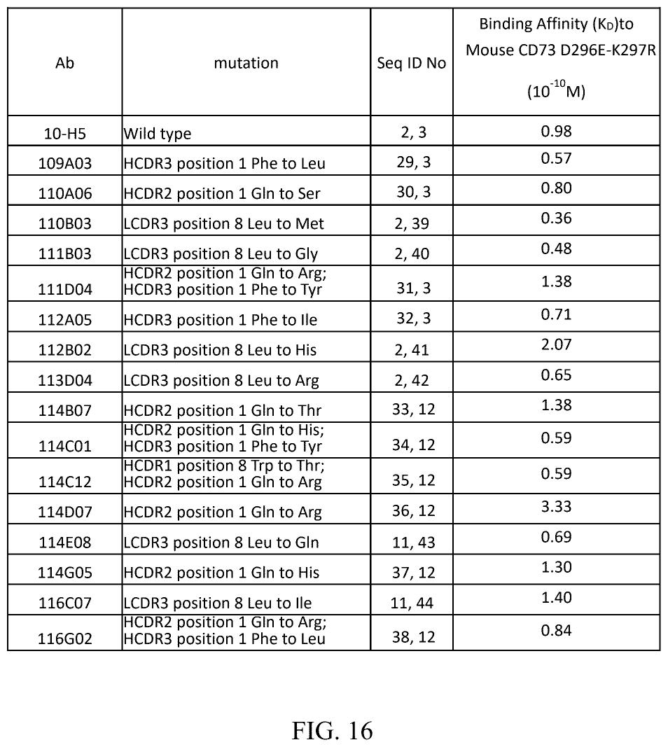

To screen more antibodies that (i) can bind to human CD73 (E296/R297) at the same amino acids as 10H5 antibody, and (ii) can inhibit the activity of CD73, the six amino acids (i.e., W33 of CDR-H1, N35 of CDR-H1, Q50 of CDR-H2, F95 of CDR-H3, Y91 of CDR-L3 and L96 of CDR-L3) were randomly mutated to construct a single-chain phage display library. Sixteen phage clones (i.e., 109A03, 110A06, 110B03, 111B03, 111D04, 112A05, 112B02, 113D04, 114B07, 114C01, 114C12, 114D07, 114E08, 114G05, 116C07, 116G02) were selected out by binding to the mouse CD73 antigen with D296E-K297R mutation and the heavy chain and the light chain variable domains were constructed into full-length IgG4 antibodies. The mutation position and the sequence ID No. of the variable domain either in heavy chain or in light chain of the 16 phage clones are shown in FIG. 16. The binding affinities (K_{D}) of the aforesaid 16 antibodies to mouse CD73 with D296E-K297R mutation were respectively analyzed by ELISA and were shown in FIG. 16.

10-4. Inhibitory Effect of Antibodies, which Selected from the Library Constructed According to the 3D Model, on the CD73 Activity

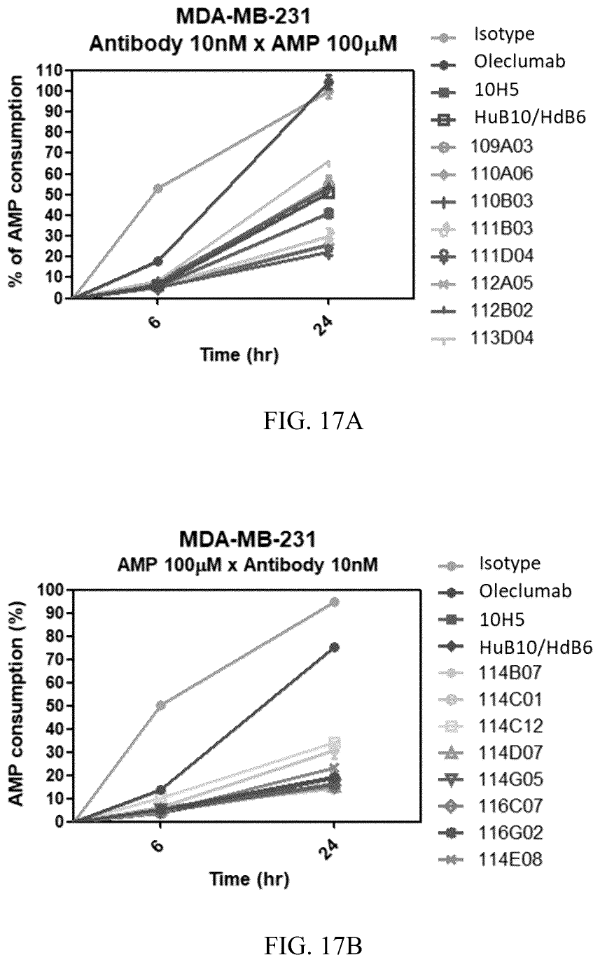

In this experiment, the detection of the inhibitory effect on CD73 activity method was the same as Example 1 by using MDA-MB-231 cancer cells, except that (i) the concentration of antibodies was 10 nM, and (ii) the concentration of AMP was 100 μM. Luminometer was used for measuring the luminescence of each group after the cells of each group were incubated for 6 hours and 24 hours. The results were shown in FIG. 17A (109A03, 110A06, 110B03, 111B03, 111D04, 112A05, 112B02, 113D04,) and FIG. 17B (114B07, 114C01, 114C12, 114D07, 114E08, 114G05, 116C07, 116G02).

As shown in FIGS. 17A and 17B, after 6 hours of culturing, both the “Oleclumab” and the antibodies of the present invention (i.e., 109A03, 110A06, 110B03, 111B03, 111D04, 112A05, 112B02, 113D04, 114B07, 114C01, 114C12, 114D07, 114E08, 114G05, 116C07, 116G02) can inhibit the consumption of AMP, wherein the antibodies of the present inventions have a better inhibitory effect than Oleclumab. On the other hand, after 24 hours of culturing, the “Oleclumab” cannot inhibit the consumption of AMP. However, the antibodies of the present inventions (i.e., 109A03, 110A06, 110B03, 111B03, 111D04, 112A05, 112B02, 113D04, 114B07, 114C01, 114C12, 114D07, 114E08, 114G05, 116C07, 116G02) can still significantly inhibit the consumption of AMP. These results indicate that, as compared to the Oleclumab, the antibodies of the present invention have a better and longer inhibitory effect on CD73 activity on MDA-MB-231 cancer cells.

1. An antibody or an antigen-binding fragment thereof, comprising:
a heavy chain variable domain comprising a HCDR1 region, a HCDR2 region and a HCDR3 region, wherein the HCDR1 region comprises an amino acid sequence of SEQ ID NO: 4 with at most one variation, the HCDR2 region comprises an amino acid sequence of SEQ ID NO: 5 with at most one variation, and the HCDR3 region comprises an amino acid sequence of SEQ ID NO: 6 with at most one variation; and
a light chain variable domain comprising a LCDR1 region, a LCDR2 region and a LCDR3 region, wherein the LCDR1 region comprises an amino acid sequence of SEQ ID NO: 7 with at most one variation, the LCDR2 region comprises an amino acid sequence of SEQ ID NO: 8 with at most one variation, and the LCDR3 region comprises an amino acid sequence of SEQ ID NO: 9 with at most one variation.

2. The antibody or antigen-binding fragment thereof according to claim 1, wherein the LCDR1 region comprises an amino acid sequence of SEQ ID NO: 7, the LCDR2 region comprises an amino acid sequence of SEQ ID NO: 8, and the LCDR3 region comprises an amino acid sequence of SEQ ID NO: 9 with at most one variation.

3. The antibody or antigen-binding fragment thereof according to claim 2, wherein:
the HCDR1 region comprises an amino acid sequence of SEQ ID NO: 4 with variation at position 8;
the HCDR2 region comprises an amino acid sequence of SEQ ID NO: 5 with variation at position 1;
the HCDR3 region comprises an amino acid sequence of SEQ ID NO: 6 with variation at position 1; and
the LCDR3 region comprises an amino acid sequence of SEQ ID NO: 9 with variations at position 8.

4. The antibody or antigen-binding fragment thereof according to claim 3, wherein:
the HCDR1 region comprises an amino acid sequence of SEQ ID NO: 4, wherein the tryptophan residue at position 8 is substituted by threonine;
the HCDR2 region comprises an amino acid sequence of SEQ ID NO: 5, wherein the glutamine residue at position 1 is substituted by serine, arginine, threonine and histidine;
the HCDR3 region comprises an amino acid sequence of SEQ ID NO: 6, wherein the phenylalanine residue at position 1 is substituted by leucine, tyrosine or isoleucine; and
the LCDR3 region comprises an amino acid sequence of SEQ ID NO: 9, wherein the leucine residue at position 8 is substituted by, methionine, glycine, histidine, arginine, glutamine and isoleucine.

5. The antibody or antigen-binding fragment thereof according to claim 1, wherein:
the HCDR1 region comprises an amino acid sequence selected from the group consisting of SEQ ID NO: 4 and SEQ ID NO: 15; and
the HCDR2 region comprises an amino acid sequence selected from the group consisting of SEQ ID NO: 5 and SEQ ID NO: 16 to SEQ ID NO: 19; and
the HCDR3 region comprises an amino acid sequence selected from the group consisting of SEQ ID NO: 6, SEQ ID NO: 20 to SEQ ID NO: 22.

6. The antibody or antigen-binding fragment thereof according to claim 1, wherein:
the LCDR1 region comprises an amino acid sequence of SEQ ID NO: 7;
the LCDR2 region comprises an amino acid sequence of SEQ ID NO: 8; and
the LCDR3 region comprises an amino acid sequence selected from the group consisting of SEQ ID NO: 9 and SEQ ID NO: 23 to SEQ ID NO: 28

7. The antibody or antigen-binding fragment thereof according to claim 1, wherein the heavy chain variable domain comprises an amino acid sequence selected from the group consisting of:
an amino acid sequence having at least 95% identity to SEQ ID NO: 2;
an amino acid sequence having at least 95% identity to SEQ ID NO: 10; and
an amino acid sequence having at least 95% identity to SEQ ID NO: 11.

8. The antibody or antigen-binding fragment thereof according to claim 1, wherein the light chain variable domain comprises an amino acid sequence selected from the group consisting of:
an amino acid sequence having at least 95% identity to SEQ ID NO: 3;
an amino acid sequence having at least 95% identity to SEQ ID NO: 12; and
an amino acid sequence having at least 95% identity to SEQ ID NO: 13.

9. The antibody or antigen-binding fragment thereof according to claim 7, wherein the heavy chain variable domain comprises an amino acid sequence selected from the group consisting of SEQ ID NO: 2, SEQ ID NO: 10, SEQ ID NO: 11 and SEQ ID NO: 29 to SEQ ID NO: 38.

10. The antibody or antigen-binding fragment thereof according to claim 8, wherein the light chain variable domain comprises an amino acid sequence selected from the group consisting of SEQ ID NO: 3, SEQ ID NO: 12, SEQ ID NO: 13 and SEQ ID NO: 39 to SEQ ID NO:44.

11. The antibody or antigen-binding fragment thereof according to claim 1, which further comprises one or more of (1) a linker peptide between the heavy chain variable domain and the light chain variable domain, (2) a heavy chain constant region, (3) a light chain constant region, and (4) an Fc region.

12. The antibody or antigen-binding fragment thereof according to claim 1, which is a single chain antibody fragment, a bispecific antibody, a single-domain antibody, a nanobody, a chimeric antibody, or a partially or fully humanized antibody.

13. The antibody or antigen-binding fragment thereof according to claim 1, which further links with a drug conjugate to form an antibody-drug conjugate (ADC), or further links with a second antibody or a second antigen-binding fragment to form a bispecific antibody.

14. The antibody or antigen-binding fragment thereof according to claim 1, which further comprises a fragment derived from IgG1, IgG2, IgG3 or IgG4.

15. The antibody or an antigen-binding fragment thereof according to claim 1, which specifically binds to CD73.

16. The antibody or antigen-binding fragment thereof according to claim 15, wherein the CD73 comprises an amino acid sequence of SEQ ID NO: 14.

17. The antibody or antigen-binding fragment thereof according to claim 15, wherein the CD73 comprises an amino acid sequence of SEQ ID NO: 1.

18. The antibody or antigen-binding fragment thereof according to claim 15, which binds to the CD73 on at least one of the glutamic acid residue at position 296 and the arginine residue at position 297.

19. The antibody or antigen-binding fragment thereof according to claim 15, which binds to the CD73 on both the glutamic acid residue at position 296 and the arginine residue at position 297.

20. An antibody or an antigen-binding fragment thereof, wherein the antibody or antigen-binding fragment thereof specifically binds to human CD73 on at least one of the glutamic acid residue at position 296 and the arginine residue at position 297.

21. The antibody or antigen-binding fragment thereof according to claim 20, which binds to human CD73 on both the glutamic acid residue at position 296 and the arginine residue at position 297.

22. The antibody or antigen-binding fragment thereof according to claim 20, wherein the CD73 comprises an amino acid sequence of SEQ ID NO: 14.

23. The antibody or antigen-binding fragment thereof according to claim 20, wherein the CD73 comprises an amino acid sequence of SEQ ID NO: 1.

24. The antibody or antigen-binding fragment thereof according to claim 20, which is a single chain antibody fragment, a single-domain antibody, a nanobody, a chimeric antibody, or a partially or fully humanized antibody.

25. The antibody or antigen-binding fragment thereof according to claim 20, which further links with a drug conjugate to form an antibody-drug conjugate (ADC), or further links with a second antibody or antigen binding fragment to form a bispecific antibody.

26. The antibody or antigen-binding fragment thereof according to claim 20, which further comprises a fragment derived from IgG1, IgG2, IgG3 or IgG4.

27. A pharmaceutical composition comprising (i) an antibody or antigen-binding fragment thereof according to claim 1, a nucleic acid molecule encoding the aforesaid antibody or antigen-binding fragment thereof, a vector comprising the aforesaid nucleic acid molecule, a recombinant host cell comprising the aforesaid nucleic acid molecule, or a recombinant host cell comprising the aforesaid vector, and (ii) a pharmaceutically acceptable carrier.

28. The pharmaceutical composition according to claim 27, which is used for inhibiting CD73.

29. The pharmaceutical composition according to claim 27, which further comprises one or more other immunotherapy agents.

30. The pharmaceutical composition according to claim 29, wherein the other immunotherapy agent is PD-1 antagonist, PD-L1 antagonist or CTLA-4 antagonist.

31. The pharmaceutical composition according to claim 27, which is used for activating T-cells, activating B-cells, activating NK cells and/or inhibiting cancer cells.

32. The pharmaceutical composition according to claim 27, which is used for treating, ameliorating and/or preventing cancer.

33. The pharmaceutical composition according to claim 32, wherein the cancer is selected from the group consisting of breast cancer, gastric cancer, colorectal cancer, gallbladder cancer, prostate cancer, ovarian carcinoma, chronic or acute lymphocytic leukemia, bladder cancer, brain tumor, kidney carcinoma, head and neck squamous cell carcinoma, glioblastoma, esophageal cancer, pancreatic cancer, renal carcinoma, oral cancer, lung cancer, colon adenocarcinoma, melanoma and lymphoma.

34. A method of inhibiting CD73, comprising administering a subject in need thereof an effective amount of an antibody or antigen-binding fragment thereof according to claim 1 or a pharmaceutical composition according to claim 27.

35. The method according to claim 34, wherein the method further comprises administering one or more other immunotherapy agents to the subject in need thereof.

36. The method according to claim 35, wherein the other immunotherapy agent is PD-1 antagonist, PD-L1 antagonist or CTLA-4 antagonist.

37. The method according to claim 34, which is for activating T-cells, activating B-cells, activating NK cells and/or inhibiting cancer cells.

38. The method according to claim 34, which is for treating, ameliorating and/or preventing cancer.

39. The method according to claim 38, wherein the cancer is selected from the group consisting of breast cancer, gastric cancer, colorectal cancer, gallbladder cancer, prostate cancer, ovarian carcinoma, chronic or acute lymphocytic leukemia, bladder cancer, brain tumor, kidney carcinoma, head and neck squamous cell carcinoma, glioblastoma, esophageal cancer, pancreatic cancer, renal carcinoma, oral cancer, lung cancer, colon adenocarcinoma, melanoma and lymphoma.


SEQUENCE LISTING (44 sequences)

SEQ ID NO: 1 - protein, 555 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHADETFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSHHHHHH

SEQ ID NO: 2 - protein, 112 aa, Mus musculus
QVKLQQSGAELVKPGASVKISCKASGYVFSSYWINWVKQRPGKGLEWIGQIYPGDGNTNYNRNFKGKATLTADKSSSTAYMQLSSLTSEDSAVYFCAGFADWGQGTTVTVSS

SEQ ID NO: 3 - protein, 107 aa, Mus musculus
DIELTQSPSSLSASLGERVSLTCRASQDIGDSLIWLQQEPDGTIKRLIYATSSLDSGVPKRFSGSRSGTSYSLTISSVEAEDAATYYCQQYSGYPLTFGSGTKLEIK

SEQ ID NO: 4 - protein, 10 aa, Mus musculus
GYVFSSYWIN

SEQ ID NO: 5 - protein, 17 aa, Mus musculus
QIYPGDGNTNYNRNFKG

SEQ ID NO: 6 - skipped

SEQ ID NO: 7 - protein, 11 aa, Mus musculus
RASQDIGDSLI

SEQ ID NO: 8 - protein, 7 aa, Mus musculus
ATSSLDS

SEQ ID NO: 9 - protein, 9 aa, Mus musculus
QQYSGYPLT

SEQ ID NO: 10 - protein, 112 aa, synthetic construct
  REGION 1..112: note = Humanized antibody
QVQLVQSGAEVKKPGASVKVSCKASGYVFSSYWINWVRQAPGQGLEWMGQIYPGDGNTNYNRNFKGRATLTADKSTSTAYMELSSLRSEDTAVYFCAGFADWGQGTLVTVSS

SEQ ID NO: 11 - protein, 112 aa, synthetic construct
  REGION 1..112: note = Humanized antibody
QVQLVQSGAEVKKPGASVKVSCKASGYVFSSYWINWVRQAPGQGLEWMGQIYPGDGNTNYNRNFKGRVTLTADKSTSTAYMELSSLRSEDTAVYYCAGFADWGQGTLVTVSS

SEQ ID NO: 12 - protein, 107 aa, synthetic construct
  REGION 1..107: note = Humanized antibody
DIQLTQSPSSLSASVGDRVTITCRASQDIGDSLIWLQQKPGKAIKRLIYATSSLDSGVPSRFSGSRSGTDYTLTISSVQPEDFATYYCQQYSGYPLTFGQGTKVEIK

SEQ ID NO: 13 - protein, 107 aa, synthetic construct
  REGION 1..107: note = Humanized antibody
DIQLTQSPSSLSASVGDRVTITCRASQDIGDSLIWLQQKPGKAPKRLIYATSSLDSGVPSRFSGSRSGTDYTLTISSLQPEDFATYYCQQYSGYPLTFGQGTKVEIK

SEQ ID NO: 14 - protein, 36 aa, Homo sapiens
SDDGRKVPVVQAYAFGKYLGYLKIEFDERGNVISSH

SEQ ID NO: 15 - protein, 10 aa, synthetic construct
  REGION 1..10: note = HCDR1
GYVFSSYTIN

SEQ ID NO: 16 - protein, 17 aa, synthetic construct
  REGION 1..17: note = HCDR2
SIYPGDGNTNYNRNFKG

SEQ ID NO: 17 - protein, 17 aa, synthetic construct
  REGION 1..17: note = HCDR2
RIYPGDGNTNYNRNFKG

SEQ ID NO: 18 - protein, 17 aa, synthetic construct
  REGION 1..17: note = HCDR2
TIYPGDGNTNYNRNFKG

SEQ ID NO: 19 - protein, 17 aa, synthetic construct
  REGION 1..17: note = HCDR2
HIYPGDGNTNYNRNFKG

SEQ ID NO: 20-22 - skipped (3 entries)

SEQ ID NO: 23 - protein, 9 aa, synthetic construct
  REGION 1..9: note = LCDR3
QQYSGYPMT

SEQ ID NO: 24 - protein, 9 aa, synthetic construct
  REGION 1..9: note = LCDR3
QQYSGYPGT

SEQ ID NO: 25 - protein, 9 aa, synthetic construct
  REGION 1..9: note = LCDR3
QQYSGYPHT

SEQ ID NO: 26 - protein, 9 aa, synthetic construct
  REGION 1..9: note = LCDR3
QQTSGYPRT

SEQ ID NO: 27 - protein, 9 aa, synthetic construct
  REGION 1..9: note = LCDR3
QQYSGYPQT

SEQ ID NO: 28 - protein, 9 aa, synthetic construct
  REGION 1..9: note = LCDR3
QQYSGYPIT

SEQ ID NO: 29 - protein, 112 aa, synthetic construct
  REGION 1..112: note = Heavy chain variable domain
QVKLQQSGAELVKPGASVKISCKASGYVFSSYWINWVKQRPGKGLEWIGQIYPGDGNTNYNRNFKGKATLTADKSSSTAYMQLSSLTSEDSAVYFCAGLADWGQGTTVTVSS

SEQ ID NO: 30 - protein, 112 aa, synthetic construct
  REGION 1..112: note = Heavy chain variable domain
QVKLQQSGAELVKPGASVKISCKASGYVFSSYWINWVKQRPGKGLEWIGSIYPGDGNTNYNRNFKGKATLTADKSSSTAYMQLSSLTSEDSAVYFCAGFADWGQGTTVTVSS

SEQ ID NO: 31 - protein, 112 aa, synthetic construct
  REGION 1..112: note = Heavy chain variable domain
QVKLQQSGAELVKPGASVKISCKASGYVFSSYWINWVKQRPGKGLEWIGRIYPGDGNTNYNRNFKGKATLTADKSSSTAYMQLSSLTSEDSAVYFCAGYADWGQGTTVTVSS

SEQ ID NO: 32 - protein, 112 aa, synthetic construct
  REGION 1..112: note = Heavy chain variable domain
QVKLQQSGAELVKPGASVKISCKASGYVFSSYWINWVKQRPGKGLEWIGQIYPGDGNTNYNRNFKGKATLTADKSSSTAYMQLSSLTSEDSAVYFCAGIADWGQGTTVTVSS

SEQ ID NO: 33 - protein, 112 aa, synthetic construct
  REGION 1..112: note = Heavy chain variable domain
QVQLVQSGAEVKKPGASVKVSCKASGYVFSSYWINWVRQAPGQGLEWMGTIYPGDGNTNYNRNFKGRVTLTADKSTSTAYMELSSLRSEDTAVYYCAGFADWGQGTLVTVSS

SEQ ID NO: 34 - protein, 112 aa, synthetic construct
  REGION 1..112: note = Heavy chain variable domain
QVQLVQSGAEVKKPGASVKVSCKASGYVFSSYWINWVRQAPGQGLEWMGHIYPGDGNTNYNRNFKGRVTLTADKSTSTAYMELSSLRSEDTAVYYCAGYADWGQGTLVTVSS

SEQ ID NO: 35 - protein, 112 aa, synthetic construct
  REGION 1..112: note = Heavy chain variable domain
QVQLVQSGAEVKKPGASVKVSCKASGYVFSSYTINWVRQAPGQGLEWMGRIYPGDGNTNYNRNFKGRVTLTADKSTSTAYMELSSLRSEDTAVYYCAGFADWGQGTLVTVSS

SEQ ID NO: 36 - protein, 112 aa, synthetic construct
  REGION 1..112: note = Heavy chain variable domain
QVQLVQSGAEVKKPGASVKVSCKASGYVFSSYWINWVRQAPGQGLEWMGRIYPGDGNTNYNRNFKGRVTLTADKSTSTAYMELSSLRSEDTAVYYCAGFADWGQGTLVTVSS

SEQ ID NO: 37 - protein, 112 aa, synthetic construct
  REGION 1..112: note = Heavy chain variable domain
QVQLVQSGAEVKKPGASVKVSCKASGYVFSSYWINWVRQAPGQGLEWMGHIYPGDGNTNYNRNFKGRVTLTADKSTSTAYMELSSLRSEDTAVYYCAGFADWGQGTLVTVSS

SEQ ID NO: 38 - protein, 112 aa, synthetic construct
  REGION 1..112: note = Heavy chain variable domain
QVQLVQSGAEVKKPGASVKVSCKASGYVFSSYWINWVRQAPGQGLEWMGRIYPGDGNTNYNRNFKGRVTLTADKSTSTAYMELSSLRSEDTAVYYCAGLADWGQGTLVTVSS

SEQ ID NO: 39 - protein, 107 aa, synthetic construct
  REGION 1..107: note = Light chain variable domain
DIELTQSPSSLSASLGERVSLTCRASQDIGDSLIWLQQEPDGTIKRLIYATSSLDSGVPKRFSGSRSGTSYSLTISSVEAEDAATYYCQQYSGYPMTFGSGTKLEIK

SEQ ID NO: 40 - protein, 107 aa, synthetic construct
  REGION 1..107: note = Light chain variable domain
DIELTQSPSSLSASLGERVSLTCRASQDIGDSLIWLQQEPDGTIKRLIYATSSLDSGVPKRFSGSRSGTSYSLTISSVEAEDAATYYCQQYSGYPGTFGSGTKLEIK

SEQ ID NO: 41 - protein, 107 aa, synthetic construct
  REGION 1..107: note = Light chain variable domain
DIELTQSPSSLSASLGERVSLTCRASQDIGDSLIWLQQEPDGTIKRLIYATSSLDSGVPKRFSGSRSGTSYSLTISSVEAEDAATYYCQQYSGYPHTFGSGTKLEIK

SEQ ID NO: 42 - protein, 107 aa, synthetic construct
  REGION 1..107: note = Light chain variable domain
DIELTQSPSSLSASLGERVSLTCRASQDIGDSLIWLQQEPDGTIKRLIYATSSLDSGVPKRFSGSRSGTSYSLTISSVEAEDAATYYCQQYSGYPRTFGSGTKLEIK

SEQ ID NO: 43 - protein, 107 aa, synthetic construct
  REGION 1..107: note = Light chain variable domain
DIQLTQSPSSLSASVGDRVTITCRASQDIGDSLIWLQQKPGKAIKRLIYATSSLDSGVPSRFSGSRSGTDYTLTISSVQPEDFATYYCQQYSGYPQTFGQGTKVEIK

SEQ ID NO: 44 - protein, 107 aa, synthetic construct
  REGION 1..107: note = Light chain variable domain
DIQLTQSPSSLSASVGDRVTITCRASQDIGDSLIWLQQKPGKAIKRLIYATSSLDSGVPSRFSGSRSGTDYTLTISSVQPEDFATYYCQQYSGYPITFGQGTKVEIK